(19) United States
(12) Patent Application Publication
(10) Pub. No.: US 2022/0240579 A1
(43) Pub. Date: Aug. 4, 2022

(54) VAPORIZER DEVICE WITH IMPROVED WICK SATURATION

(71) Applicant: JUUL Labs, lnc., San Francisco, CA (US)

(72) Inventors: Matthew Czapar, San Francisco, CA (US); Conrad Leqendy, San Francisco, CA (US); Christopher Loental, San Francisco, CA (US)

(21) Appl. No.: 17/620,637

(22) PCT Filed: Jun. 17, 2020
(86) PCT No.: PCT/US20/38212

§ 371 Date: Dec. 17, 2021

Related U.S. Application Data
(60) Provisional application No. 62/863,150, filed on Jun. 18, 2019.

(51) Int. Cl. A24F 40/44; A24F 40/51; A24F 40/57; A24F 40/10

(52) U.S. Cl. CPC A24F 40/44; A24F 40/51; A61M 21/02; A24F 40/10; A24F 40/57

(57) ABSTRACT

Features relating to a vaporizer device including a reusable vaporizer device body and a cartridge with a reservoir containing a vaporizable material are provided. Aspects of the current subject matter provide for improved wick saturation by reducing the viscosity of the vaporizable material and aiding in the release of air bubbles that form and are trapped in the wick as the vaporizable material is consumed by a user, thereby improving aerosol production. The improved wick saturation is achieved by applying, between user puffs on the vaporizer device, a mechanical agitation and/or a standby temperature setting.

### CROSS-REFERENCE TO RELATED APPLICATIONS

This application claims priority to U.S. Provisional Patent Application No. 62/863,150, filed Jun. 18, 2019 and entitled “Vaporizer Device with Improved Wick Saturation,” the content of which is incorporated in its entirety by reference herein.

### TECHNICAL FIELD

The current subject matter described herein relates generally to vaporizer devices, such as portable, personal vaporizer devices for generating and delivering an inhalable aerosol from one or more vaporizable materials, and more particularly relates to wick saturation in a vaporizer device.

### BACKGROUND

Vaporizing devices, including electronic vaporizers or e-vaporizer devices, allow the delivery of vapor and aerosol containing one or more active ingredients by inhalation of the vapor and aerosol. Electronic vaporizer devices are gaining increasing popularity both for prescriptive medical use, in delivering medicaments, and for consumption of nicotine, tobacco, other liquid-based substances, and other plant-based smokeable materials, such as cannabis, including solid (e.g., loose-leaf or flower) materials, solid/liquid (e.g., suspensions, liquid-coated) materials, wax extracts, and prefilled pods (cartridges, wrapped containers, etc.) of such materials. Electronic vaporizer devices in particular may be portable, self-contained, and convenient for use.

### SUMMARY

Aspects of the current disclosure relate to saturation of a wick in a cartridge of a vaporizer device for improved aerosol production.

According to an aspect of the current subject matter, a vaporizer device includes at least one data processor and at least one memory storing instructions which, when executed by the at least one data processor, cause operations including detecting an end of a user puff on a cartridge of the vaporizer device, the cartridge including a vaporizable material, and applying, in response to the detection of the end of the user puff, one or more of a standby temperature setting to a heating element of the vaporizer device and a mechanical agitation by a haptics system of the vaporizer device. The heating element is in thermal contact with a wick configured to convey from a reservoir the vaporizable material to a part of the cartridge in which the heating element is contained, where the vaporizable material is vaporized by heat delivered from the heating element.

According to an inter-related aspect, a method includes detecting an end of a user puff on a cartridge of a vaporizer device, the cartridge including a vaporizable material, and applying, in response to the detection of the end of the user puff, one or more of a standby temperature setting to a heating element of the vaporizer device and a mechanical agitation by a haptics system of the vaporizer device. The heating element is in thermal contact with a wick configured to convey from a reservoir the vaporizable material to a part of the cartridge in which the heating element is contained, where the vaporizable material is vaporized by heat delivered from the heating element.

According to an inter-related aspect, a non-transitory computer readable medium is provided, the non-transitory computer readable medium storing instructions, which when executed by at least one data processor, result in operations including detecting an end of a user puff on a cartridge of a vaporizer device, the cartridge including a vaporizable material, and applying, in response to the detection of the end of the user puff, one or more of a standby temperature setting to a heating element of the vaporizer device and a mechanical agitation by a haptics system of the vaporizer device. The heating element is in thermal contact with a wick configured to convey from a reservoir the vaporizable material to a part of the cartridge in which the heating element is contained, where the vaporizable material is vaporized by heat delivered from the heating element.

According to an inter-related aspect, an apparatus includes means for detecting an end of a user puff on a cartridge of a vaporizer device, the cartridge including a vaporizable material, and means for applying, in response to the detection of the end of the user puff, one or more of a standby temperature setting to a heating element of the vaporizer device and a mechanical agitation by a haptics system of the vaporizer device. The heating element is in thermal contact with a wick configured to convey from a reservoir the vaporizable material to a part of the cartridge in which the heating element is contained, where the vaporizable material is vaporized by heat delivered from the heating element.

In some variations, one or more of the features disclosed herein including the following features can optionally be included in any feasible combination. The instructions, when executed, may further cause operations of detecting a user puff on the cartridge, and enabling the heating element of the vaporizer device to reach a vaporization temperature. The standby temperature setting may provide a standby temperature of the heating element that is lower than the vaporization temperature and higher than an ambient temperature. Re-saturation settings may define one or more characteristics of the standby temperature setting and/or one or more characteristics of the mechanical agitation. The one or more characteristics of the standby temperature setting and/or the one or more characteristics of the mechanical agitation may be based on properties of the vaporizable material. The one or more characteristics of the standby temperature setting may include a length of time for the standby temperature setting to be applied. The one or more characteristics of the mechanical agitation may include one or more of a defined frequency, a defined duty cycle, a defined time duration, and a defined intensity. The re-saturation settings may be based on a maximum viscosity value. The re-saturation settings may be based on user preferences, a type of the vaporizable material being consumed, properties of the vaporizable material, an amount of the vaporizable material remaining in the cartridge, an age of the vaporizable material, ambient characteristics, characteristics internal to the cartridge, use data, properties of the wick, properties of the heating element, or a combination thereof. The use data may include a number of puffs taken from the cartridge, a duration of the puffs taken, time the puffs were taken, time between successive puffs, an amount of total particulate matter generated by the puffs taken, or a combination thereof. The instructions, when executed, may further cause operations of receiving, through the cartridge, the re-saturation settings. The re-saturation settings may be determined such that a total particulate matter generation for second and subsequent puffs is within a predefined range of a first total particulate matter generation for a first puff. The one or more of the standby temperature setting and the mechanical agitation may be consistent with a power or battery setting defined by a user or predefined by the vaporizer device. The haptics system may include an actuator, a linear resonant actuator, an eccentric rotating mass motor, or a combination thereof. The one or more of the standby temperature setting and the mechanical agitation may be applied in between user puffs at a predetermined schedule of frequency. The instructions, when executed, may further cause operations of receiving, through a user setting, an application setting, a remote database, the cartridge, or a combination thereof, information relating to the vaporizable material. The information relating to the vaporizable material may include at least a viscosity of the vaporizable material, and one or more characteristics of the standby temperature setting and/or one or more characteristics of the mechanical agitation may be provided via one or more look-up tables utilizing the viscosity of the vaporizable material. The one or more look-up tables may be accessible through memory of the vaporizer device, a controller of the vaporizer device via signaling with one or more remote devices, or a combination thereof.

The details of one or more variations of the subject matter described herein are set forth in the accompanying drawings and the description below. Other features and advantages of the subject matter described herein will be apparent from the description and drawings, and from the claims. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings:

FIG. 1A-FIG. 1F illustrate features of a vaporizer device including a vaporizer body and a cartridge consistent with implementations of the current subject matter;

FIG. 2 is a schematic block diagram illustrating features of a vaporizer device having a cartridge and a vaporizer body consistent with implementations of the current subject matter;

FIG. 3 illustrates communication between a vaporizer device, a user device, and a server consistent with implementations of the current subject matter;

FIG. 4A-FIG. 4B illustrate internal features of a vaporizer body and a cartridge consistent with implementations of the current subject matter;

FIG. 5 illustrates features of a cartridge of a vaporizer device consistent with implementations of the current subject matter;

FIG. 6 illustrates, via a cross-sectional view, features of a cartridge of a vaporizer device consistent with implementations of the current subject matter;

FIG. 7 depicts a block diagram illustrating an example of proportional-integral-derivative (PID) control consistent with implementations of the current subject matter;

FIG. 8A-FIG. 8C illustrate pressure aspects related to a cartridge and a wick of a vaporizer device consistent with implementations of the current subject matter;

FIG. 9A and FIG. 9B illustrate viscosity properties related to flow rate and temperature consistent with implementations of the current subject matter;

FIG. 10A and FIG. 10B illustrate total particulate matter properties with respect to time and heat of a wick of a vaporizer device consistent with implementations of the current subject matter;

FIG. 11 illustrates temperature properties with respect to time of a heating coil of a traditional vaporizer device;

FIG. 12 illustrates total particulate matter properties with respect to time of a wick of a traditional vaporizer device;

FIG. 13 illustrates temperature properties with respect to time of a heating coil of a vaporizer device consistent with implementations of the current subject matter;

FIG. 14 illustrates total particulate matter properties with respect to time of a wick of a vaporizer device consistent with implementations of the current subject matter;

FIG. 15A and FIG. 15B illustrate saturation properties with respect to total particulate matter and viscosity of a wick of a vaporizer device consistent with implementations of the current subject matter; and

FIG. 16A and FIG. 16B are flowcharts illustrating processes of operating the vaporizer device consistent with implementations of the current subject matter.

When practical, similar reference numbers denote similar structures, features, or elements.

### DETAILED DESCRIPTION

Aspects of the current subject matter relate to saturation of a wick configured to absorb vaporizable material contained in a cartridge of a vaporizer device. In some implementations, a heater in the cartridge includes a heating element such as a heating coil or other resistive or inductive heating element that is in thermal contact with the wick. The wick may be formed from one or more materials capable of causing passive fluid motion (e.g., by capillary action and/or the like) to convey a quantity of the vaporizable material to a part of the cartridge in which the heating element is contained. That is, the wick may draw the vaporizable material from a reservoir in the cartridge to the heating element such that the vaporizable material may be vaporized by heat delivered from the heating element for subsequent emission from the vaporizer device, for instance, for inhalation by a user in a gas and/or a condensed (e.g., aerosol particles or droplets) phase.

Wick saturation is directly correlated with aerosol production. Thus, adequate saturation of the wick of the vaporizable material aids in providing a user a consistent and desired experience. The greater the variability in the saturation of the wick when a user puffs on the vaporizer device, the greater the variability in the amount of aerosol produced by the vaporizer device, which may lead to an inconsistent, unsatisfying, and/or undesirable user experience. Aspects of the current subject matter provide for saturation of the wick for improved aerosol production.

Before providing additional details regarding aspects of wick saturation in the cartridge, the following provides a description of some examples of vaporizer devices including a vaporizer body and a cartridge in which aspects of the current subject matter may be implemented. The following descriptions are meant to be exemplary, and wick saturation processes consistent with the current subject matter are not limited to the example vaporizer devices described herein.

Implementations of the current subject matter include devices relating to vaporizing of one or more materials for inhalation by a user. The term “vaporizer” may be used generically in the following description and may refer to a vaporizer device, such as an electronic vaporizer. Vaporizers consistent with the current subject matter may be referred to by various terms such as inhalable aerosol devices, aerosolizers, vaporization devices, electronic vaping devices, electronic vaporizers, vape pens, etc. Examples of vaporizers consistent with implementations of the current subject matter include electronic vaporizers, electronic cigarettes, e-cigarettes, or the like. In general, such vaporizers are often portable, hand-held devices that heat a vaporizable material to provide an inhalable dose of the material. The vaporizer may include a heater configured to heat a vaporizable material which results in the production of one or more gas-phase components of the vaporizable material. A vaporizable material may include liquid and/or oil-type plant materials, or a semi-solid like a wax, or plant material such as leaves or flowers, either raw or processed. The gas-phase components of the vaporizable material may condense after being vaporized such that an aerosol is formed in a flowing air stream that is deliverable for inhalation by a user. The vaporizers may, in some implementations of the current subject matter, be particularly adapted for use with an oil-based vaporizable material, such as cannabis-derived oils although other types of vaporizable materials may be used as well.

One or more features of the current subject matter, including one or more of a cartridge (also referred to as a vaporizer cartridge or pod) and a reusable vaporizer device body (also referred to as a vaporizer device base, a body, a vaporizer body, or a base), may be employed with a suitable vaporizable material (where suitable refers in this context to being usable with a device whose properties, settings, etc. are configured or configurable to be compatible for use with the vaporizable material). The vaporizable material may include one or more liquids, such as oils, extracts, aqueous or other solutions, etc., of one or more substances that may be desirably provided in the form of an inhalable aerosol. The cartridge may be inserted into the vaporizer body, and then the vaporizable material heated which results in the inhalable aerosol.

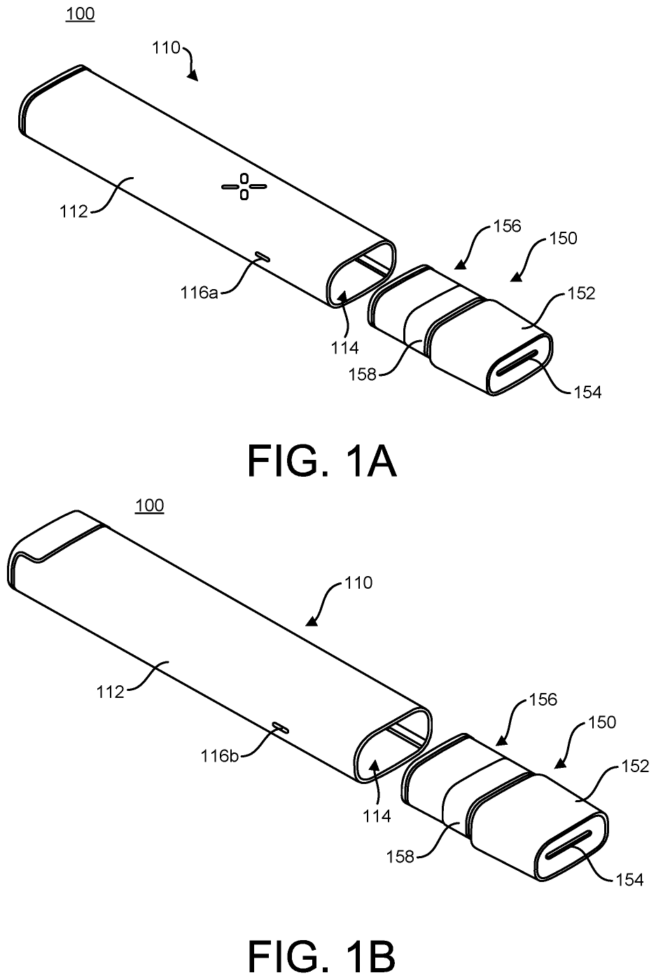

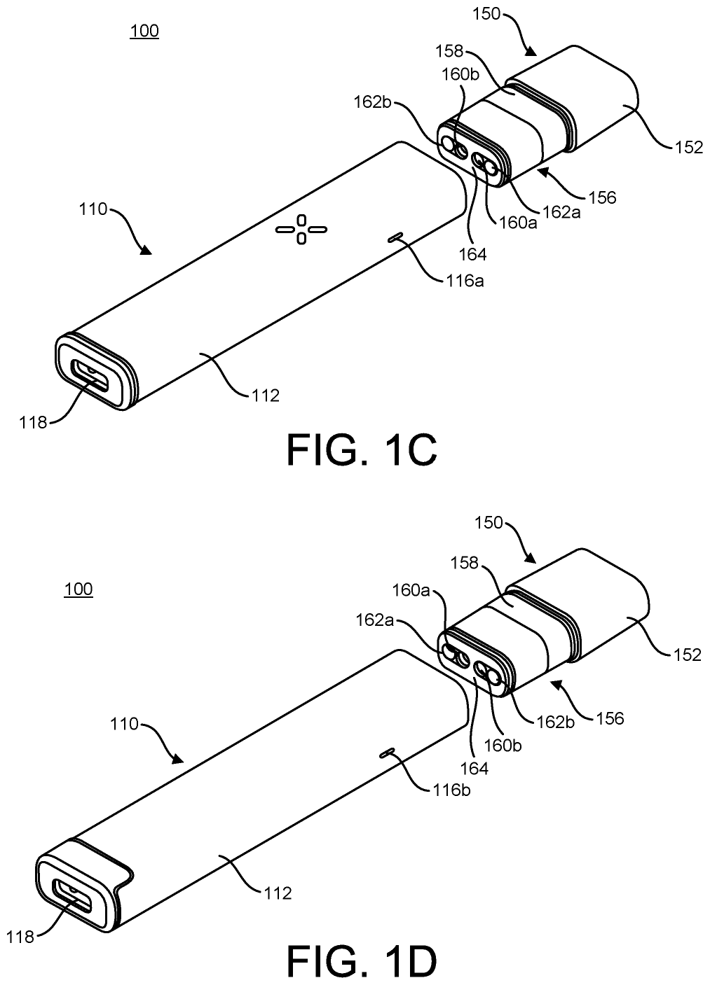

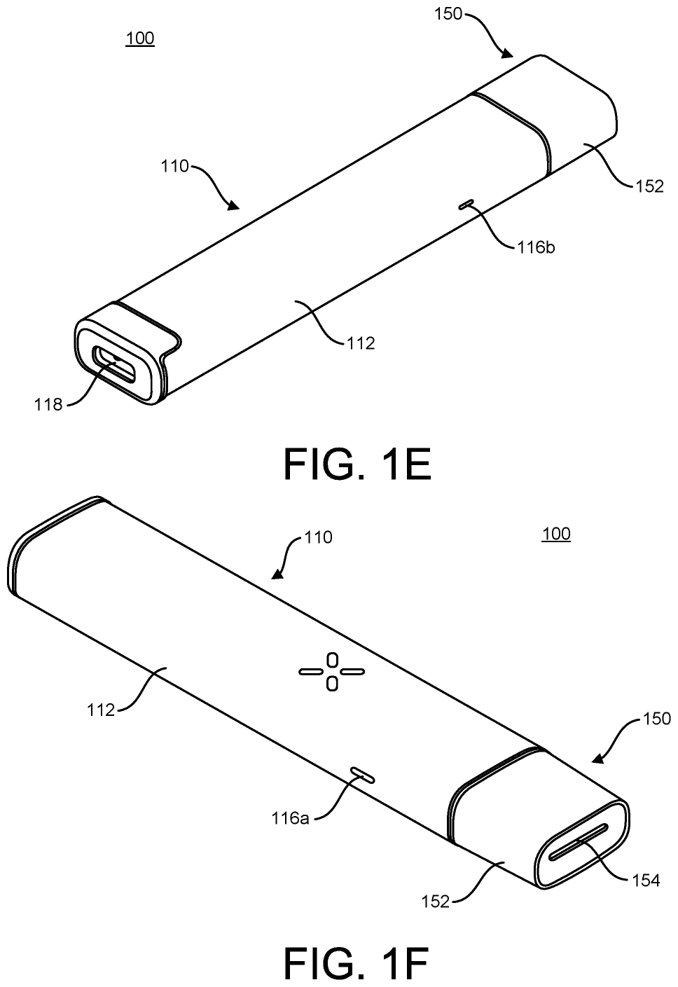

FIG. 1A-FIG. 1F illustrates features of a vaporizer device 100 including a vaporizer body 110 and a cartridge 150 consistent with implementations of the current subject matter. FIG. 1A is a bottom perspective view, and FIG. 1B is a top perspective view of the vaporizer device 100 with the cartridge 150 separated from a cartridge receptacle 114 on the vaporizer body 110. Both of the views in FIG. 1A and FIG. 1B are shown looking towards a mouthpiece 152 of the cartridge 150. FIG. 1C is a bottom perspective view, and FIG. 1D is a top perspective view of the vaporizer device with the cartridge 150 separated from the cartridge receptacle 114 of the vaporizer body 110. FIG. 1C and FIG. 1D are shown looking toward the distal end of the vaporizer body 110. FIG. 1E is top perspective view, and FIG. 1F is a bottom perspective view of the vaporizer device 100 with the cartridge 150 engaged for use with the vaporizer body 110.

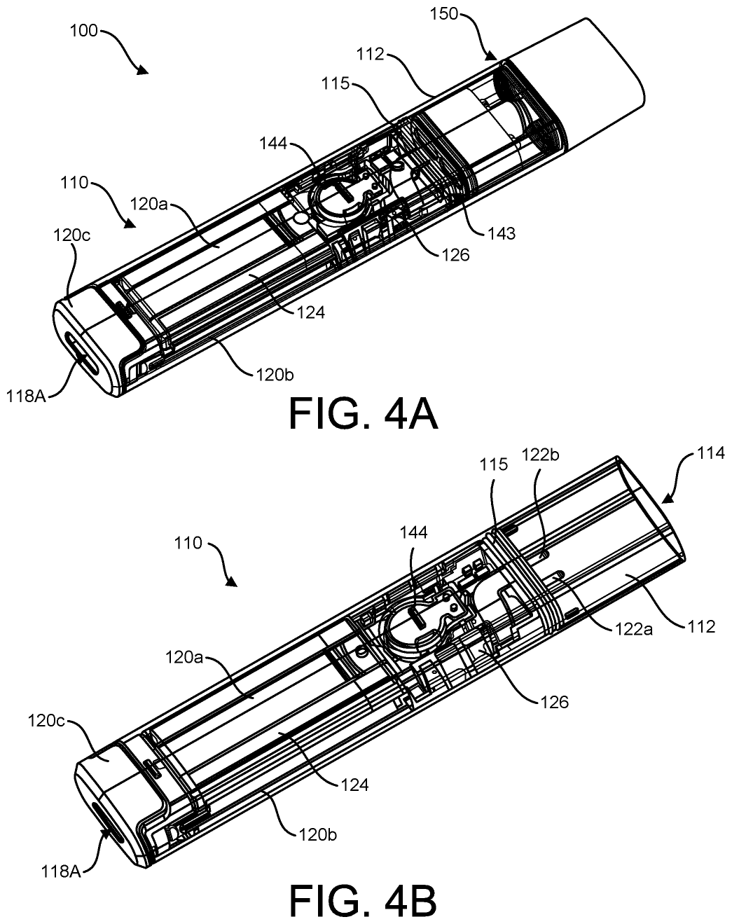

As shown in FIG. 1A-FIG. 1D, the cartridge 150 includes, at the proximal end, a mouthpiece 152 that is attached over a cartridge body 156 that forms a reservoir or tank 158 that holds a vaporizable material. The cartridge body 156 may be transparent, translucent, opaque, or a combination thereof. The mouthpiece 152 may include one or more openings 154 (see FIG. 1A, FIG. 1B, FIG. 1F) at the proximal end out of which vapor may be inhaled, by drawing breath through the vaporizer device 100. The distal end of the cartridge body 156 may couple to and be secured to the vaporizer body 110 within the cartridge receptacle 114 of the vaporizer body 110. Power pin receptacles 160a,b (see FIG. 1C, FIG. 1D) of the cartridge 150 mate with respective power pins or contacts 122a,b (see, for example, FIG. 4B) of the vaporizer body 110 that extend into the cartridge receptacle 114. The cartridge 150 also includes air flow inlets 162a,b on the distal end of the cartridge body 156.

A tag 164, such as a data tag, a near-field communication (NFC) tag, or other type of wireless transceiver or communication tag, may be positioned on at least a portion of the distal end of the cartridge body 156. As shown in FIG. 1C and FIG. 1D, the tag 164 may substantially surround the power pin receptacles 160a,b and the air flow inlets 162a,b, although other configurations of the tag 164 may be implemented as well. For example, the tag 164 may be positioned between the power pin receptacle 160a and the power pin receptacle 160b, or the tag 164 may be shaped as a circle, partial circle, oval, partial oval, or any polygonal shape encircling or partially encircling the power pin receptacles 160a,b and the air flow inlets 162a,b or a portion thereof.

In the example of FIG. 1A, the vaporizer body 110 has an outer shell or cover 112 that may be made of various types of materials, including for example aluminum (e.g., AL6063), stainless steel, glass, ceramic, titanium, plastic (e.g., Acrylonitrile Butadiene Styrene (ABS), Nylon, Polycarbonate (PC), Polyethersulfone (PESU), and the like), fiberglass, carbon fiber, and any hard, durable material. The proximal end of the vaporizer body 110 includes an opening forming the cartridge receptacle 114, and the distal end of the vaporizer body 110 includes a connection 118, such as, for example, a universal serial bus Type C (USB-C) connection and/or the like. The cartridge receptacle 114 portion of the vaporizer body 110 includes one or more openings (air inlets) 116a,b that extend through the outer shell 112 to allow airflow therein, as described in more detail below. The vaporizer body 110 as shown has an elongated, flattened tubular shape that is curvature-continuous, although the vaporizer body 110 is not limited to such a shape. The vaporizer body 110 may take the form of other shapes, such as, for example, a rectangular box, a cylinder, and the like.

The cartridge 150 may fit within the cartridge receptacle 114 by a friction fit, snap fit, and/or other types of secure connection. The cartridge 150 may have a rim, ridge, protrusion, and/or the like for engaging a complimentary portion of the vaporizer body 110. While fitted within the cartridge receptacle 114, the cartridge 150 may be held securely within but still allow for being easily withdrawn to remove the cartridge 150.

Although FIG. 1A-FIG. 1F illustrate a certain configuration of the vaporizer device 100, the vaporizer device 100 may take other configurations as well.

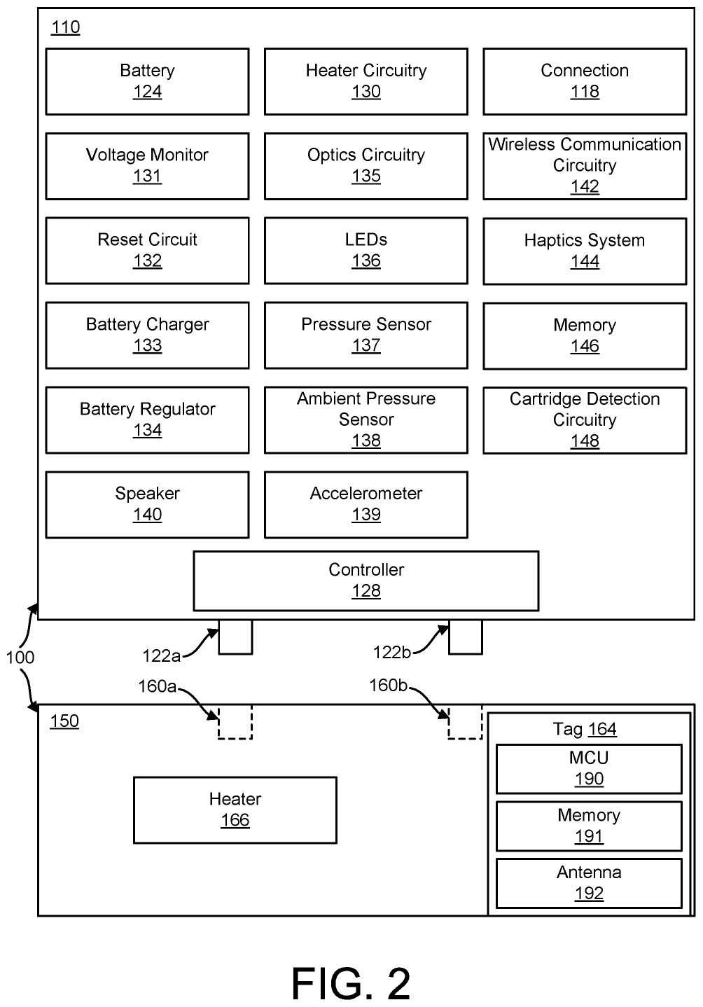

FIG. 2 is a schematic block diagram illustrating components of the vaporizer device 100 having the cartridge 150 and the vaporizer body 110 consistent with implementations of the current subject matter. Included in the vaporizer body 110 is a controller 128 that includes at least one processor and/or at least one memory configured to control and manage various operations among the components of the vaporizer device 100 described herein.

Heater control circuitry 130 of the vaporizer body 110 controls a heater 166 of the cartridge 150. The heater 166 may generate heat to provide vaporization of the vaporizable material. For example, the heater 166 may include a heating coil (e.g., a resistive heater) in thermal contact with a wick which absorbs the vaporizable material, as described in further detail below.

A battery 124 is included in the vaporizer body 110, and the controller 128 may control and/or communicate with a voltage monitor 131 which includes circuitry configured to monitor the battery voltage, a reset circuit 132 configured to reset (e.g., shut down the vaporizer device 100 and/or restart the vaporizer device 100 in a certain state), a battery charger 133, and a battery regulator 134 (which may regulate the battery output, regulate charging/discharging of the battery, and provide alerts to indicate when the battery charge is low, etc.).

The power pins 122a,b (see also FIG. 4B) of the vaporizer body 110 engage the complementary power pin receptacles 160a,b of the cartridge 150 when the cartridge 150 is engaged with the vaporizer body 110. Alternatively, power pins may be part of the cartridge 150 for engaging complementary power pin receptacles of the vaporizer body 110. The engagement allows for the transfer of energy from an internal power source (e.g., the battery 124) to the heater 166 in the cartridge 150. The controller 128 may regulate the power flow (e.g., an amount or current and/or a voltage amount) to control a temperature at which the heater 166 heats the vaporizable material contained in the reservoir 158. According to implementations of the current subject matter, a variety of electrical connectors other than a pogo-pin and complementary pin receptacle configuration may be used to electrically connect the vaporizer body 110 and the cartridge 150, such as for example, a plug and socket connector.

The controller 128 may control and/or communicate with optics circuitry 135 (which controls and/or communicates with one or more displays such as LEDs 136 which can provide user interface output indications), a pressure sensor 137, an ambient pressure sensor 138, an accelerometer 139, and/or a speaker 140 configured to generate sound or other feedback to a user.

The pressure sensor 137 may be configured to sense a user drawing (e.g., inhaling) on the mouthpiece 152 and activate the heater control circuitry 130 of the vaporizer body 110 to accordingly control the heater 166 of the cartridge 150. In this way, the amount of current supplied to the heater 166 may be varied according the user's draw (e.g., additional current may be supplied during a draw, but reduced when there is not a draw taking place). The ambient pressure sensor 138 may be included for atmospheric reference to reduce sensitivity to ambient pressure changes and may be utilized to reduce false positives potentially detected by the pressure sensor 137 when measuring draws from the mouthpiece 152.

The accelerometer 139 (and/or other motion sensors, capacitive sensors, flow sensors, strain gauge(s), or the like) may be used to detect user handling and interaction, for example, to detect movement of the vaporizer body 110 (such as, for example, tapping, rolling, and/or any other deliberate movement associated with the vaporizer body 110). The detected movements may be interpreted by the controller 128 as one or more predefined user commands. For example, one particular movement may be a user command to gradually increase the temperature of the heater 166 as the user intends to begin using the vaporizer device 100.

The vaporizer body 110, as shown in FIG. 2, includes wireless communication circuitry 142 that is connected to and/or controlled by the controller 128. The wireless communication circuitry 142 may include a near-field communication (NFC) antenna that is configured to read from and/or write to the tag 164 of the cartridge 150. Alternatively or additionally, the wireless communication circuitry 142 may be configured to automatically detect the cartridge 150 as it is being inserted into the vaporizer body 110. In some implementations, data exchanges between the vaporizer body 110 and the cartridge 150 take place over NFC. In some implementations, data exchanges between the vaporizer body 110 and the cartridge 150 may take place via a wired connection such as various wired data protocols.

The wireless communication circuitry 142 may include additional components including circuitry for other communication technology modes, such as Bluetooth circuitry, Bluetooth Low Energy circuitry, Wi-Fi circuitry, cellular (e.g., LTE, 4G, and/or 5G) circuitry, and associated circuitry (e.g., control circuitry), for communication with other devices. For example, the vaporizer body 110 may be configured to wirelessly communicate with a remote processor (e.g., a smartphone, a tablet, a computer, wearable electronics, a cloud server, and/or processor based devices) through the wireless communication circuitry 142, and the vaporizer body 110 may through this communication receive information including control information (e.g., for setting temperature, resetting a dose counter, etc.) from and/or transmit output information (e.g., dose information, operational information, error information, temperature setting information, charge/battery information, etc.) to one or more of the remote processors.

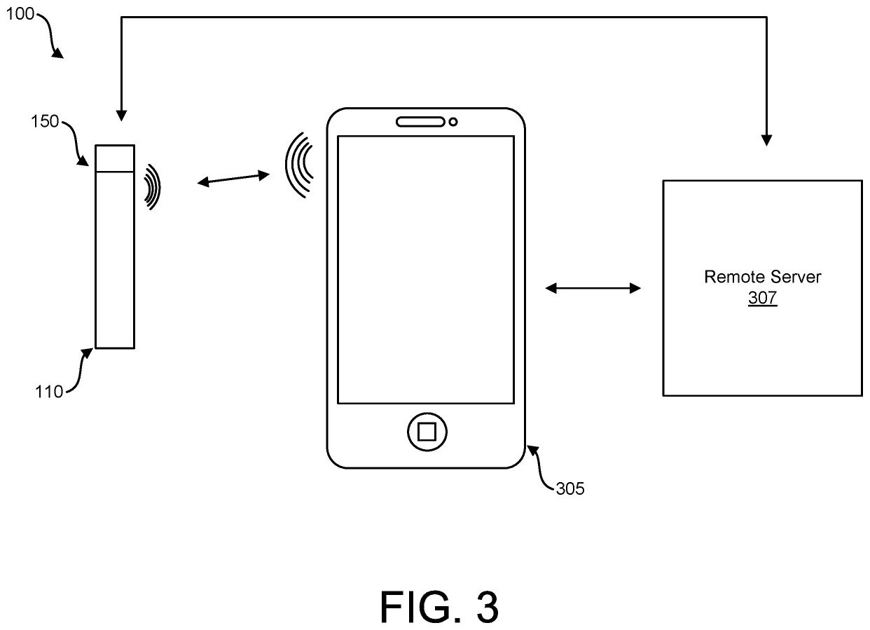

The tag 164 may be a type of wireless transceiver and may include a microcontroller unit (MCU) 190, a memory 191, and an antenna 192 (e.g., an NFC antenna) to perform the various functionalities described below with further reference to FIG. 3. The tag 164 may be, for example, a 1 Kbit or a 2 Kbit tag that is of type ISO/IEC 15693. NFC tags with other specifications may also be used. The tag 164 may be implemented as active NFC, enabling reading and/or writing information via NFC with other NFC compatible devices including a remote processor, another vaporizer device, and/or wireless communication circuitry 142. Alternatively, the tag 164 may be implemented using passive NFC technology, in which case other NFC compatible devices (e.g., a remote processor, another vaporizer device, and/or wireless communication circuitry 142) may only be able to read information from the tag 164.

The vaporizer body 110 may include a haptics system 144, such as an actuator, a linear resonant actuator (LRA), an eccentric rotating mass (ERM) motor, or the like that provide haptic feedback such as a vibration as a “find my device” feature or as a control or other type of user feedback signal. For example, using an app running on a user device (such as, for example, a user device 305 shown in FIG. 3), a user may indicate that he/she cannot locate his/her vaporizer device 100. Through communication via the wireless communication circuitry 142, the controller 128 sends a signal to the haptics system 144, instructing the haptics system 144 to provide haptic feedback (e.g., a vibration). The controller 128 may additionally or alternatively provide a signal to the speaker 140 to emit a sound or series of sounds. The haptics system 144 and/or speaker 140 may also provide control and usage feedback to the user of the vaporizer device 100; for example, providing haptic and/or audio feedback when a particular amount of a vaporizable material has been used or when a period of time since last use has elapsed. Alternatively or additionally, haptic and/or audio feedback may be provided as a user cycles through various settings of the vaporizer device 100. Alternatively or additionally, the haptics system 144 and/or speaker 140 may signal when a certain amount of battery power is left (e.g., a low battery warning and recharge needed warning) and/or when a certain amount of vaporizable material remains (e.g., a low vaporizable material warning and/or time to replace the cartridge 150). Alternatively or additionally, the haptics system 144 and/or speaker 140 may also provide usage feedback and/or control of the configuration of the vaporizer device 100 (e.g., allowing the change of a configuration, such as target heating rate, heating rate, etc.).

The vaporizer body 110 may include circuitry for sensing/detecting when a cartridge 150 is connected and/or removed from the vaporizer body 110. For example, cartridge-detection circuitry 148 may determine when the cartridge 150 is connected to the vaporizer body 110 based on an electrical state of the power pins 122a,b within the cartridge receptacle 114. For example, when the cartridge 150 is present, there may be a certain voltage, current, and/or resistance associated with the power pins 122a,b, when compared to when the cartridge 150 is not present. Alternatively or additionally, the tag 164 may also be used to detect when the cartridge 150 is connected to the vaporizer body 110.

The vaporizer body 110 also includes the connection (e.g., USB-C connection, micro-USB connection, and/or other types of connectors) 118 for coupling the vaporizer body 110 to a charger to enable charging the internal battery 124. Alternatively or additionally, electrical inductive charging (also referred to as wireless charging) may be used, in which case the vaporizer body 110 would include inductive charging circuitry to enable charging. The connection 118 at FIG. 2 may also be used for a data connection between a computing device and the controller 128, which may facilitate development activities such as, for example, programming and debugging.

The vaporizer body 110 may also include a memory 146 that is part of the controller 128 or is in communication with the controller 128. The memory 146 may include volatile and/or non-volatile memory or provide data storage. In some implementations, the memory 146 may include 8 Mbit of flash memory, although the memory is not limited to this and other types of memory may be implemented as well.

FIG. 3 illustrates communication between the vaporizer device 100 (including the vaporizer body 110 and the cartridge 150), the user device 305 (e.g., a smartphone, tablet, laptop, desktop computer, a workstation, and/or the like), and a remote server 307 (e.g., a server coupled to a network, a cloud server coupled to the Internet, and/or the like) consistent with implementations of the current subject matter. The user device 305 wirelessly communicates with the vaporizer device 100. A remote server 307 may communicate directly with the vaporizer device 100 or through the user device 305. The vaporizer body 110 may communicate with the user device 305 and/or the remote server 307 through the wireless communication circuitry 142. In some implementations, the cartridge 150 may establish through the tag 164 communication with the vaporizer body 110, the user device 305, and/or the remote server 307. While the user device 305 in FIG. 3 is depicted as a type of handheld mobile device, the user device 305 consistent with implementations of the current subject matter is not so limited and may be, as indicated, various other types of user computing devices.

An application software (“app”) running on at least one of the remote processors (the user device 305 and/or the remote server 307) may be configured to control operational aspects of the vaporizer device 100 and receive information relating to operation of the vaporizer device 100. For example, the app may provide a user with capabilities to input or set desired properties or effects, such as, for example, a particular temperature or desired dose, which is then communicated to the controller 128 of the vaporizer body 110 through the wireless communication circuitry 142. The app may also provide a user with functionality to select one or more sets of suggested properties or effects that may be based on the particular type of vaporizable material in the cartridge 150. For example, the app may allow adjusting heating based on the type of vaporizable material, the user's (of the vaporizer device 100) preferences or desired experience, and/or the like. The app may be a mobile app and/or a browser-based or web app. For example, the functionality of the app may be accessible through one or more web browsers running on one or more types of user computing devices.

Data read from the tag 164 from the wireless communication circuitry 142 of the vaporizer body 110 may be transferred to one or more of the remote processors (e.g., the user device 305 and/or the remote server 307) to which it is connected, which allows for the app running on the one or more processors to access and utilize the read data for a variety of purposes. For example, the read data relating to the cartridge 150 may be used for providing recommended temperatures, dose control, usage tracking, and/or assembly information.

The cartridge 150 may also communicate directly, through the tag 164, with other devices. This enables data relating to the cartridge 150 to be written to/read from the tag 164, without interfacing with the vaporizer body 110. The tag 164 thus allows for identifying information (e.g., pod ID, batch ID, etc.) related to the cartridge 150 to be associated with the cartridge 150 by one or more remote processors. For example, when the cartridge 150 is filled with a certain type of vaporizable material, this information may be transmitted to the tag 164 by filling equipment. Then, the vaporizer body 110 is able to obtain this information from the tag 164 (e.g., via the wireless communication circuitry 142 at the vaporizer body 110) to identify the vaporizable material currently being used and accordingly adjust the controller 128 based on, for example, user-defined criteria or pre-set parameters associated with the particular type of vaporizable material (set by a manufacturer or as determined based upon user experiences/feedback aggregated from other users). For example, a user may establish (via the app) a set of criteria relating to desired effects for or usage of one or more types of vaporizable materials. When a certain vaporizable material is identified, based on communication via the tag 164, the controller 128 may accordingly adopt the established set of criteria, which may include, for example, temperature and dose, for that particular vaporizable material.

As described above, the vaporizer device 100 and/or the user device 305 that is part of a vaporizer system as defined above may include a user interface (e.g., including an app or application software) that may be executed on the user device 305 in communication, which may be configured to determine, display, enforce, and/or meter dosing.

The vaporizer device 100 consistent with implementations of the current subject matter may be configured to facilitate social interaction through the vaporizer device 100. For example, the vaporizer device 100 may be configured to share usage information with others, such as third parties including health care providers, etc., for better prescription and administration of medical treatment. The vaporizer device 100 may also be configured to communicate with non-medical third parties (e.g., friends, colleagues, etc.), and with unknown third parties (making some or all information publically available). In some implementations, the vaporizer device 100 described herein, either by itself or in communication with one or more communications devices that are part of a system, may identify and provide information about the operation, status, or user input from the vaporizer device 100 to a public or private network.

Software, firmware, or hardware that is separate or separable from the vaporizer device and that wirelessly communicates with the vaporizer device 100 may be provided as described with respect to FIG. 3. For example, applications (“apps”) may be executed on a processor of a desktop device or station and/or a portable and/or wearable device, including smartphones, smartwatches, and the like, which may be referred to as a personal digital device, a user device, or optionally just a device (e.g., user device 305 in FIG. 3) that is part of a connected system. The user device 305 may provide an interface for the user to engage and interact with functions related to the vaporizer device 100, including communication of data to and from the vaporizer device 100 to the user device 305 and/or additional third party processor (e.g., servers such as the remote server 307 in FIG. 3). For example, a user may control some aspects of the vaporizer device 100 (temperature, session size, etc.) and/or data transmission and data receiving to and from the vaporizer device 100, optionally over a wireless communication channel between first communication hardware of the user device 305 and second communication hardware of the vaporizer device 100. Data may be communicated in response to one or more actions of the user (e.g., including interactions with a user interface displayed on the user device 305), and/or as a background operation such that the user does not have to initiate or authorize the data communication process.

User interfaces may be deployed on the user device 305 and may aid the user in operating the vaporizer device 100. For example, the user interface operating on the user device 305 may include icons and text elements that may inform the user of various ways that settings may be adjusted or configured by the user. In this manner (or in others consistent with the current subject matter) information about the vaporizer device 100 may be presented using a user interface displayed by the user device 305. Icons and/or text elements may be provided to allow the user to see information regarding one or more statuses of the vaporizer device 100, such as battery information (charge remaining, draws remaining, time to charge, charging, etc.), cartridge status (e.g., type of cartridge and vaporizable material, fill status of cartridge, etc.), and other device statuses or information. Icons and/or text elements may be provided to allow the user to update internal software (a.k.a., firmware) in the vaporizer device 100. Icons and text elements may be provided to allow the user to set security and/or authorization features of the vaporizer device 100, such as setting a PIN code to activate the vaporizer device 100 or the use of personal biometric information as a way of authentication. Icons and text elements may be provided to allow the user to configure foreground data sharing and related settings.

The vaporizer device 100 may perform onboard data gathering, data analysis, and/or data transmission methods. As mentioned, the vaporizer device 100 having wired or wireless communication capability may interface with digital consumer technology products such as smart phones, tablet computers, laptop/netbook/desktop computers, wearable wireless technologies such as “smart watches,” and other wearable technology such as Google “Glass,” or similar through the use of programming, software, firmware, GUI, wireless communication, wired communication, and/or software commonly referred to as application(s) or “apps.” A wired communication connection may be used to interface the vaporizer device 100 to digital consumer technology products for the purpose of the transmission and exchange of data to/from the vaporizer device from/to the digital consumer technology products (and thereby also interfacing with apps running on the digital consumer technology products). A wireless communication connection may be used to interface the vaporizer device 100 to digital consumer technology products for the transmission and exchange of data to/from the vaporizer device 100 from/to the digital wireless interface. The vaporizer device may use a wireless interface that includes one or more of an infrared (IR) transmitter, a Bluetooth interface, an 802.11 specified interface, and/or communications with a cellular telephone network in order to communicate with consumer technology.

FIG. 4A and FIG. 4B provide partial internal views of the vaporizer body 110 in an assembled configuration consistent with implementations of the current subject matter. FIG. 4A and FIG. 4B are top perspective views of the vaporizer body 110. The cartridge 150 is shown inserted into the cartridge receptacle 114 in FIG. 4A, while FIG. 4B illustrates the vaporizer body 110 without the cartridge 150 inserted. FIG. 4A and FIG. 4B illustrate placement of the battery 124 with respect to a printed circuit board assembly (PCBA) 126 which may combine with the power pins 122a,b, a first antenna such as an integrated near-field communication antenna 143, and additional components such as for example the connection 118 and a second antenna such as an integrated Bluetooth antenna. Also shown is connection of the haptics system 144 (e.g., a LRA) with the PCBA 126, and portions of outer structural supports 120a, 120b, and 120c (with opening 118a) and gasket 115 that may form a skeleton or support assembly. Spring contacts (such as, for example, pogo pins, although other types of pins, contacts, etc. may be used as well) on the PCBA 126 may be provided for connection with the haptics system 144. For example, the haptics system 144 may include connection pads that are configured to contact the spring contacts on a portion of the PCBA 126.

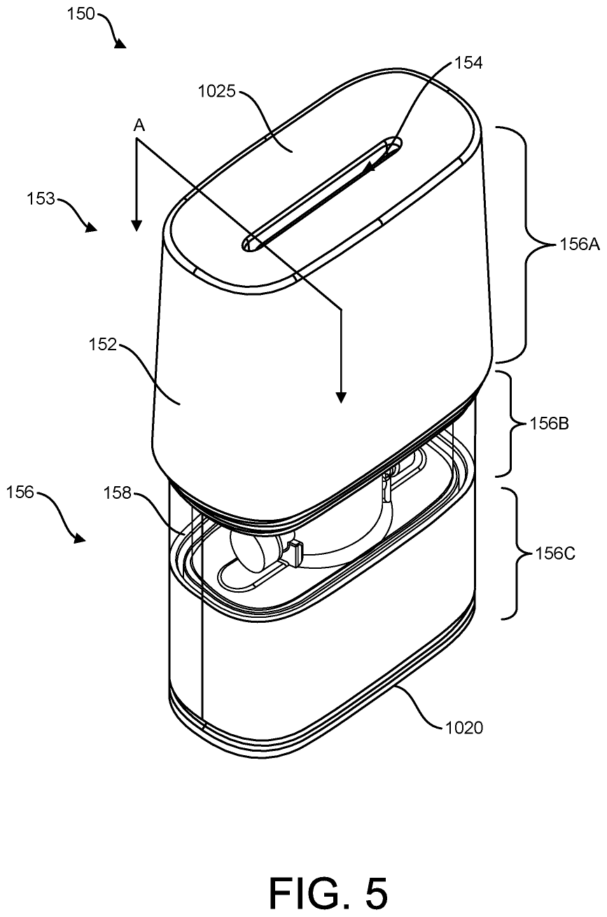

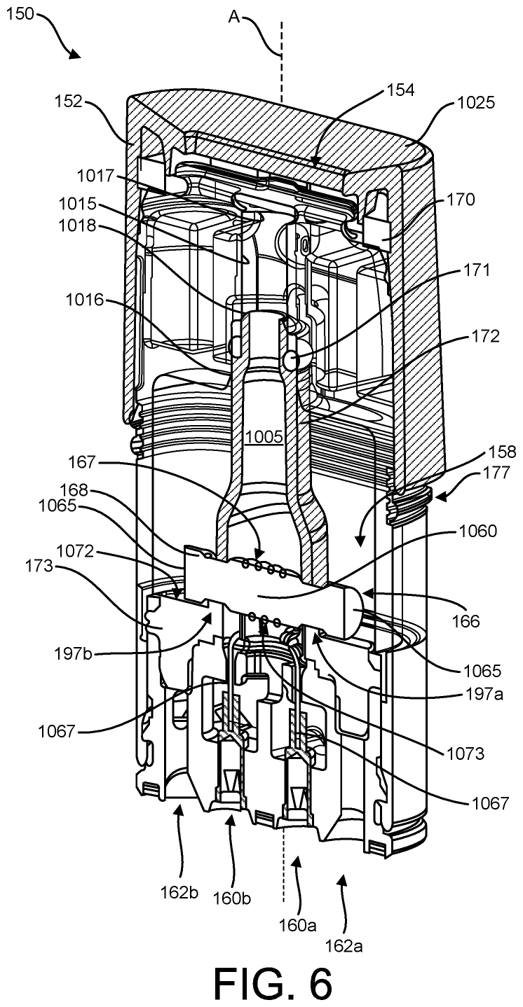

FIG. 5 and FIG. 6 illustrate exemplary features of the cartridge 150 of the vaporizer device 100 consistent with implementations of the current subject matter. The cartridge 150 may include the cartridge body 156 defining, at least in part, the reservoir 158 configured to contain vaporizable material, the mouthpiece 152, and a vaporizing assembly of vapor-generating components positioned within the cartridge body 156 and configured to vaporize the vaporizable material.

The cartridge body 156 may be divided, generally, into a proximal end region 156A, a central region 156B, and a distal end region 156C. The proximal end region 156A of the cartridge body 156 may be coupled to the mouthpiece 152 configured to deliver the vapor to the user. The central region 156B includes the tank or reservoir 158 defined, at least in part, by the cartridge body 156 and configured to contain an amount of the vaporizable material. The distal end region 156C of the cartridge body 156 may house one or more components configured to vaporize the material from the reservoir 158 into a vaporizer chamber 1005 (see FIG. 6). The mouthpiece 152 is configured to interface with the user to release the vapor from the vaporizer chamber 1005 to the user through the one or more openings 154 in the mouthpiece 152, for example, upon the user drawing a breath through the vaporizer device 100.

The upper, proximal end region 156A of the cartridge body 156 is configured to couple with the mouthpiece 152, for example, by inserting within an internal volume of the mouthpiece 152 such that an exterior surface of the cartridge body 156 near the upper proximal end region 156A seals with an inner surface of the mouthpiece 152. As such, the mouthpiece 152 may form the proximal end of the cartridge 150. The mouthpiece 152 may have an external surface that is generally amenable to a user placing their lips over a proximal end 153 of the mouthpiece 152 to inhale the vapor.

The proximal end region 156A of the cartridge body 156 can define a central channel 1015 (see FIG. 6) for directing vapor from the vaporization chamber 1005 towards the one or more openings 154 through the mouthpiece 152. The lower, distal end region 156C of the cartridge body 156 may house components configured to couple with the vaporizer body 110, for example, by inserting within the cartridge receptacle 114. The central region 156B of the cartridge body 156 positioned between the proximal and distal end regions 156A, 156C and remains hollow such that it may define, in part, the reservoir 158.

As mentioned, the distal end region 156C of the cartridge body 156 may be configured to couple to and be secured with the vaporizer body 110, for example, by inserting within the cartridge receptacle 114 (see FIG. 1A-FIG. 1F). The walls of the receptacle 114 may surround the cartridge body 156 on a distal end 1020 and all four sides of the distal end region 156C and the central region 156B. A mouthpiece seal 177 may be positioned between and configured to seal between an inner surface of the mouthpiece 152 and an outer surface of the cartridge body 156. The mouthpiece seal 177 may provide a snap-fit feel upon seating the cartridge 150 within the receptacle 114 of the vaporizer body 110.

The one or more openings 154 may extend through a proximal end surface 1025 into the internal volume of the mouthpiece 152. The one or more openings 154 allow for the vapor produced within the cartridge 150 to be inhaled by the user. The one or more openings 154 may be aligned with a central, longitudinal axis A of the cartridge 150 or positioned off-set from the longitudinal axis A. The proximal end surface 1025 of the mouthpiece 152 may be sloped inwardly away from the outer edges towards the one or more openings 154.

The internal volume of the mouthpiece 152 may include a region, for example, near the proximal end 153 of the cartridge 150 adjacent the one or more openings 154 of the mouthpiece 152, that is configured to contain one or more absorbent pads 170 within the internal volume. The one or more pads 170 may be positioned within the internal volume of the mouthpiece 152 near or proximate to the one or more openings 154 through which vapor may be inhaled, e.g., by drawing breath through the vaporizer device 100, such that it may capture moisture just prior to inhalation by the user. The one or more absorbent pads 170 may prevent or reduce the flow of fluid, such as the vaporizable material, into and out of the one or more openings 154. The one or more absorbent pads 170 may be pushed against the interior surface of the mouthpiece 152 or may be pulled away from interior walls so as to maximize the surface area available for moisture absorption.

The central region 156B defines, in part, the tank or reservoir 158 configured to hold an amount of vaporizable material within the cartridge 150. The reservoir 158 may be sealed on a distal or bottom end by an internal sealing gasket 173 positioned within the distal end region 156C of the cartridge body 156. The reservoir 158 may be sealed on a proximal or top end by a sealing ring 171. A central cannula 172 may extend through the reservoir 158 from near the distal end region 156C of the cartridge body 156 to the proximal end region 156A of the cartridge body 156. As best shown in FIG. 6, the proximal end region 156A of the cartridge body 156 defines the central channel 1015 that extends between a first opening 1016 at an upper end of the reservoir 158 to a second opening 1017 that may be coaxially aligned with the opening 154 through the proximal end surface of the mouthpiece 152. A proximal tap 1018 of the central cannula 172 encircled by the sealing ring 171 may extend through the first opening 1016 a distance into the central channel 1015. The sealing ring 171 may seal with the surface of the central channel 1015 and thereby seal the reservoir 158 on the upper end.

The enlarged base of the central cannula 172 may be coupled to a bottom plate 1072. The bottom plate 1072 may be a generally planar feature coupled to the base of the central cannula 172 that forms a rim around the base. The lower surface of the bottom plate 1072 may include distal extensions configured to extend through the internal gasket 173. The upper surface of the bottom plate 1072 may define, at least in part, a lower surface of the reservoir 158 and the lower surface of the bottom plate 1072 may abut against the internal gasket 173. The bottom plate 1072 may include a central aperture 1073 such that the vaporization chamber 1005 remains open on a distal end to provide a vapor flow passageway through the cartridge body 156 to the mouthpiece 152. The central aperture 1073 may be elongated such that it forms an oval, elliptical, or other elongate shape having a minor axis and a major axis. A middle portion of the central aperture 1073 may be aligned with the vaporization chamber 1005 and at least partially encircled by the cannula 172.

The cartridge 150 includes a vaporizing assembly of vapor-generating components. The vapor-generating components may include the heater 166 configured to heat the vaporizable material to a sufficient temperature that it may vaporize. The vapor-generating components may be arranged as an atomizer or cartomizer or oven. The vapor may be released to a vaporization chamber where the gas phase vapor may condense, forming an aerosol cloud having typical liquid vapor particles with particles having a diameter of average mass of approximately 0.1 micron or greater. In some cases, the diameter of average mass may be approximately 0.1-1 micron.

The heater 166 of the vaporizing assembly may cause the vaporizable material to be converted from a condensed form (e.g., a solid, a liquid, a solution, a suspension, a part of an at least partially unprocessed plant material, etc.) to the gas phase. After conversion of the vaporizable material to the gas phase, and depending on the type of vaporizer, the physical and chemical properties of the vaporizable material, and/or other factors, at least some of the gas-phase vaporizable material may condense to form particulate matter in at least a partial local equilibrium with the gas phase as part of an aerosol, which may form some or all of an inhalable dose provided by the vaporizer device 100 for a given puff or draw on the vaporizer device 100. It will be understood that the interplay between gas and condensed phases in an aerosol generated by a vaporizer may be complex and dynamic, as factors such as ambient temperature, relative humidity, chemistry, flow conditions in airflow paths (both inside the vaporizer and in the airways of a human or other animal), mixing of the gas-phase or aerosol-phase vaporizable material with other air streams, etc., may affect one or more physical parameters of an aerosol.

Vaporizers for use with liquid vaporizable materials (e.g., neat liquids, suspensions, solutions, mixtures, etc.) typically include an atomizer in which a wicking element (also referred to herein as a wick 168), which may include any material capable of causing passive fluid motion (for example, by capillary action) to convey an amount of a liquid vaporizable material to a part of the atomizer that includes the heating element. The wicking element is generally configured to draw liquid vaporizable material from the reservoir configured to contain (and that may in use contain) the liquid vaporizable material such that the liquid vaporizable material may be vaporized by heat delivered from the heating element.

The heater 166 may be or include one or more of a conductive heater, a radiative heater, a convective heater, a resistive heater, and/or an inductive heater. One type of vaporizing heating element is a resistive heating element, which may be constructed of or at least include a material (e.g., a metal or alloy, for example a nickel-chromium alloy, or a non-metallic resistor) configured to dissipate electrical power in the form of heat when electrical current is passed through one or more resistive segments of the heating element. In some implementations of the current subject matter, an atomizer may include a vaporizing heating element that includes a resistive coil or other heating element wrapped around, positioned within, integrated into a bulk shape of, pressed into thermal contact with, or otherwise arranged to deliver heat to a wicking element to cause a liquid vaporizable material drawn by the wicking element from a reservoir to be vaporized for subsequent inhalation by a user in a gas and/or a condensed (e.g., aerosol particles or droplets) phase.

The heater 166 may be configured to heat and/or vaporize at least a portion of the vaporizable material drawn towards the heater 166 from the reservoir 158. The central cannula 172 defining the vaporization chamber 1005 is configured to couple to the heater 166 configured to generate heat to provide vaporization of the vaporizable material contained in the reservoir 158. In some implementations, the heater 166 of the vaporizing assembly may include a resistive element such as a heating coil 167 in thermal contact with the wick 168 of the vaporizing assembly. The wick 168 may be formed of any of a variety of materials, including metals, polymer, natural fibers, synthetic fibers, or combinations of these. For example, the wick 168 may be formed of silica fibers, cotton, ceramic, hemp, stainless steel mesh, rope cables, and/or any porous medium, such as for example sintered glass beads. The wick 168 is porous and provides a capillary pathway for fluid within the reservoir 158 through and into the wick 168. The capillary pathway is generally large enough to permit wicking of sufficient material to replace vaporized liquid transferred from the tank by capillary action (wicking) during vaporization, but may be small enough to prevent leakage of the vaporizable material out of the cartridge during normal operation, including when pressure is applied to outside the cartridge 150. The wick 168 may have a size configured to handle high viscosity liquids. The wick 168 may also be configured to handle low viscosity liquids. In some implementations, the wick 168 may have a diameter that is at least about 1.5 mm. The wick may have a variety of ranges of diameter, including less than 1.5 mm and larger than 1.5 mm in diameter (e.g., about 1.0 mm or larger, about 1.1 mm or larger, about 1.2 mm or larger, about 1.3 mm or larger, about 1.4 mm or larger, about 1.6 mm or larger, about 1.7 mm or larger, about 1.8 mm or larger, about 1.9 mm or larger, about 2.0 mm or larger, about 2.1 mm or larger, about 2.2 mm or larger, about 2.3 mm or larger, about 2.4 mm or larger, about 2.5 mm or larger, etc., including between about 1.8 mm and about 5 mm, between about 1.9 mm and about 4 mm, between about 2 mm and about 4 mm, etc.). The material of the wick 168 is configured to draw the liquid vaporizable material from the reservoir 158 into the vaporization chamber 1005 without the need for a pump or other mechanical moving part. In some implementations, the tension of the heating coil 167 wound around the wick 168 may vary. Winding the heating coil 167 tighter and/or with additional windings may create a larger heating surface area to create more intense or concentrated heating of the vaporizable material. Likewise, reducing the diameter of the wick may also create more intense or concentrated heating of the vaporizable material.

The heating coil 167 may be a resistance wire wrapped around the wick 168 and connected to a positive and negative pole of a current source. The heating coil 167 may increase in temperature as a result of the current flowing through the wire to generate heat. The heat may be transferred to at least a portion of the vaporizable material through conductive, convective, and/or radiative heat transfer such that at least a portion of the vaporizable material vaporizes. Air drawn into the vaporization chamber 1005 may carry the vapor away from the heating element 166.

The heater 166 may extend across the air path within the vaporization chamber 1005, such as in a transverse direction. The central cannula 172 may be arranged coaxial with the longitudinal axis A of the device, and the wick 168 may extend orthogonal to the longitudinal axis A through the central cannula 172. The wick 168 is preferably positioned near a distal-most end region of the reservoir 158 such that the vaporizable material in the reservoir 158 may be fully used. A pair of lateral openings 1074a,b may extend through the walls of the central cannula 172 near its base where the cannula 172 couples to the bottom plate 1072. The pair of lateral openings 1074a,b may be aligned across from one another on opposing sides of the central cannula 172. The openings 1074a,b are provided and sized for coupling to the heater 166. As described above, the bottom plate 1072 and the elongate central aperture 1073 extending through the plate 1072 may have a major axis and a minor axis. The elongate shape of the central aperture 1073 provides for two outer portions along the major axis of the plate 1072 to extend beyond the base of the central cannula 172. The two outer portions of the central aperture 1073 may be aligned with the lateral openings 1074a,b of the central cannula 172 thereby forming an enlarged slot near the base of the central cannula 172 where it couples with the bottom plate 1072. The wick 168 may extend through these lateral openings 1074a,b and within this slot.

In some implementations, the wick 168 of the heater 166 may include a central portion 1060 and opposing ends 1065. The heating coil 167 may be wrapped around the central portion 1060 of the wick 168, which in turn may be positioned within the vaporization chamber 1005. The opposing ends 1065 of the wick 168 may be positioned outside the vaporization chamber 1005 by extending laterally outward through the lateral openings 1074a,b of the cannula 172. As such, the opposing ends 1065 may be positioned within the internal volume of the reservoir 158 whereas the central portion 1060 of the wick 168 wrapped by the heating coil 167 may be positioned inside the vaporization chamber 1005 of the central cannula 172. Leads 1067 of the heating coil 167 may extend away from the central portion 1060 of the wick 168 and down through the central aperture 1073 of the bottom plate 1072 out of the vaporization chamber 1005. The leads 1067 may extend into the distal end region 156C of the cartridge body 156 where the leads 1067 may electrically couple with receptacles 160a,b.

As mentioned, the distal end region 156C of the cartridge body 156 may house the internal sealing gasket 173, which may be coupled to a lower support structure 174. The gasket 173 may be positioned generally under the bottom plate 1072 of the central cannula 172 and attached to an upper surface of the lower support structure 174. This placement of the gasket 173 serves to seal the reservoir 158 on the distal or bottom end and thereby reduce or eliminate leaking of vaporizable material out of the reservoir 158, for example, into the electrical components contained in the distal end region 156C of the cartridge 150 as well as the vaporizer body 110. The gasket 173 may be, in some implementations, an oversized elastic or rubberized material that plugs various openings in a distal end region of the device and forms a seal between the reservoir 158 and the lower support structure 174 when under compression. Thus, the gasket 173 may be sized and shaped to fit between the reservoir 158 and the lower support structure 174 to seal any openings therebetween.

As described above, the wick 168 may extend orthogonal to the longitudinal axis A at the base of the reservoir 158. The opposing ends 1065 of the wick 168 may be positioned within the reservoir 158 and the central region 1060 of the wick 168 wound by the heating coil 167 may be positioned within the vaporization chamber 1005. The upper half of the wick 168 may be sealed by the walls of the central cannula 172 defining the lateral openings 1074a,b. The lower half of the wick 168 may engage and seal with surface features 197a,b of the gasket 173. The surface features 197a,b may be sized and shaped to insert through the central aperture 1073 of the bottom plate 1072 helping to seal the central aperture 1073. At least a portion of the surface features 197a,b extends a distance toward the opposing ends 1065 of the wick 168. This portion of the surface features 197a,b may include a wick mating surface sized and shape to complement the cylindrical surface of the wick 168. For example, the portion may have a semi-circular wick mating surface configured to seal with the cylindrical outer surface of a region of the wick 168. The portion of the surface features 197a,b may also laterally support the opposing ends 1065 of the wick 168.

As mentioned above, the leads 1067 of the heating coil 167 extend through the central aperture 1073 of the bottom plate 1072 as well as through a central opening of the gasket 173 into the lower support structure 174. The leads 1067 of the heating coil 167 may electrically couple with the receptacles 160a,b within the lower region 1078 of the support structure 174. The receptacles 160a,b may be power pin receptacles configured to mate with respective power pins or contacts 122a,b of the vaporizer body 110, for example, pins projecting upward from a bottom end of the receptacle 114, as described elsewhere herein. The power pins 122a,b are configured to insert into the respective receptacles 160a,b; the engagement between the power pins 122a,b and the receptacles 160a,b allowing for the transfer of energy from an internal power source of the vaporizer body 110 to the leads 1067 of the heating coil 167.

In operation, after the vaporizer device 100 is fully charged (or in some instances partially charged), a user may activate the vaporizer device 100 by drawing (e.g., inhaling) through the mouthpiece. The vaporizer device 100 may detect a draw (e.g., using one or more pressure sensors, flow sensors, microphones, and/or the like, including a sensor configured to detect a change in temperature or power applied to a heater element) and may increase the power to a predetermined temperature preset. The power may be regulated by the controller by detecting the change in resistance of the heating coil and using the temperature coefficient of resistivity to determine the temperature.

In accordance with some implementations of the current subject matter, a vaporizer device may be controlled so that the temperature used to vaporize the vaporizable material is maintained within a preset range. In general, the controller may control the temperature of the resistive heater (e.g., resistive coil, etc.) based on a change in resistance due to temperature (e.g., temperature coefficient of resistance (TCR)). For example, a heater may be any appropriate resistive heater, such as, for example, a resistive coil. The heater is typically coupled to the heater controller via two or more connectors (electrically conductive wires or lines) so that the heater controller applies power (e.g., from the power source) to the heater. The heater controller may include regulatory control logic to regulate the temperature of the heater by adjusting the applied power. The heater controller may include a dedicated or general-purpose processor, circuitry, or the like and is generally connected to the power source and may receive input from the power source to regulate the applied power to the heater.

For example, apparatuses consistent with implementations described herein may include logic for determining the temperature of the heater based on the TCR of the heating element (resistive coil), based on sensed resistance of the coil. The resistance of the heater (e.g., a resistive heater) may be measured (Rheater) and the controller may use the known properties of the heater (e.g., the temperature coefficient of resistance) for the heater to determine the temperature of the heater. For example, the resistance of the heater may be detected by a detection circuit connected at the electrical contacts that connect to the cartridge, and this resistance compared to a target resistance, which is typically the resistance of the resistive heater at the target temperature. In some cases this resistance may be estimated from the resistance of the resistive hearing element at ambient temperature (baseline).

In some example embodiments, the controller 128 may be configured to control a temperature of the heater 166 including, for example, by adjusting and/or maintaining the temperature of the heating coil 167. The temperature of the heating coil 167 may be adjusted and/or maintained by at least controlling a discharge of the battery 124 to the heating coil 167. For instance, the controller 128 may start the discharge of battery 124 to the heating coil 167 in order to raise the temperature of the heating coil 167. Alternatively or additionally, the controller 128 may stop the discharge of the battery 124 to the heating coil 167 in order to maintain and/or decrease the temperature of the heating coil 167.

According to some example embodiments, the controller 128 may apply a proportional-integral-derivative (PID) control technique when adjusting the temperature of the heating coil 167. For example, the controller 128 may adjust the temperature of the heating coil 167, including by starting or stopping the discharge of the battery 124 to the heating coil 167, based on an error in the current temperature of the heating coil 167 relative to a target temperature. It should be appreciated that the temperature of the heating coil 167 may correspond to a resistance through the heating coil 167. That is, the temperature of the heating coil 167 may be correlated to the resistance through the heating coil 167 by a thermal coefficient of resistance associated with the heating coil 167. As such, the current resistance through the heating coil 167 may correspond to the current temperature of the heating coil while the target resistance through the heating coil 167 may correspond to the target temperature of the heating coil 167. Moreover, the controller 128 may start or stop the discharge of the battery 124 to the heating coil 167 based on an error in the current resistance through the heating coil 167 relative to a target resistance.

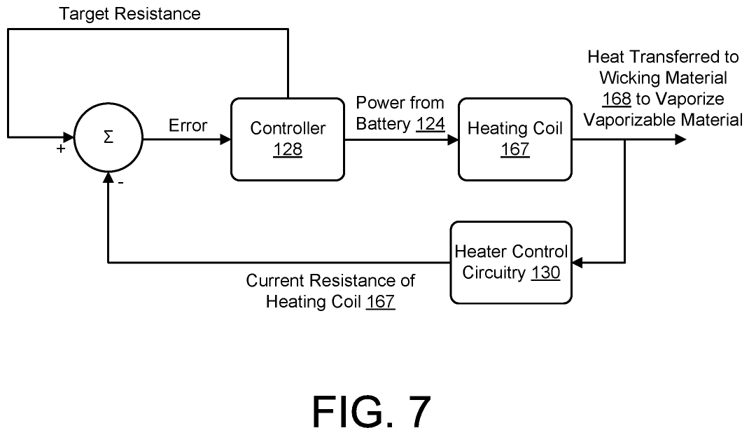

To further illustrate, FIG. 7 depicts a block diagram illustrating an example of proportional-integral-derivative (PID) control consistent with implementations of the current subject matter. As shown in FIG. 7, the controller 128 may control the discharge of the battery 124 to the heating coil 167 in the heater 166 of the cartridge 150. Meanwhile, the flow of current from the battery 124 through the heating coil 167 may generate heat, for example, through resistive heating. The heat generated by the heating coil 167 may be transferred to the wick 168, which may be in thermal contact with the heating coil 167. For instance, the heat that is generated by the heating coil 167 may be transferred to the wick 168 through conductive heat transfer, convective heat transfer, radiative heat transfer, and/or the like. The heat from the heating coil 167 may vaporize at least some of the vaporizable material held by the wick 168.

Referring again to FIG. 7, the heater control circuitry 130 may be configured to determine a current resistance of the heating coil 167. As noted, the current resistance of the heating coil 167 may correspond to a current temperature of the heating coil 167. Accordingly, the controller 128, when applying a proportional-integral-derivative control technique, may adjust and/or maintain the temperature of the heating coil 167 based at least on an error between the current resistance of the heating coil 167 and a target resistance corresponding to at target temperature for the heating coil 167. As shown in FIG. 7, the controller 128 may adjust, based at least on the error between the current resistance through the heating coil 167 and the target resistance, the discharge of the battery 124 to the heating coil 167. For example, the controller 128 may start the discharge of battery 124 to the heating coil 167 if the current resistance of the heating coil 167 is below the target resistance. Alternatively or additionally, the controller 128 may stop the discharge of the battery 124 to the heating coil 167 if the current resistance of the heating coil 167 is equal to and/or above the target resistance.

As noted elsewhere herein, wick saturation aspects of the current subject matter are not intended to be limited in use to the foregoing examples of vaporizer devices. Rather, the foregoing descriptions are provided merely as examples in which the described wick saturation aspects may be utilized. Variations of the exemplary vaporizer devices described herein may be used with the wick saturations aspects of the current subject matter. For example, variations of the cartridge may be used with the wick saturation aspects of the current subject matter. As another example, in one implementation, a single-use integrated vaporizer device, which may not include a removable cartridge, may employ the wick saturation aspects consistent with implementations of the current subject matter. In another example, a wick-based desktop vaporizer unit may also employ the wick saturation aspects. In yet another example, the wick saturation aspects of the current subject matter may be used in aromatherapy mist emitters and the like.

Additionally, while some implementations of the current subject matter may be described with respect to cannabis and cannabinoid-based vaporizable materials, for example cannabis oils, the disclosure is not limited to cannabis and cannabinoid-based vaporizable materials and may be applicable to other types of materials.

Turning to aspects of wick saturation consistent with implementations of the current subject matter, as previously described, wick saturation is correlated with aerosol production. Adequate saturation of the wick by the vaporizable material aids in providing a user a consistent and desired experience. Consistent with implementations of the current subject matter, adequate saturation as used herein refers to an increased rate of wick saturation, resulting in an incremental increase in total particulate matter. Total particulate matter refers to an amount of the vaporizable material removed from the wick (e.g., the total mass of the aerosol generated).

A greater variability in the saturation of the wick when a user puffs on the vaporizer device may correspond to a greater variability in the amount of aerosol produced by the vaporizer device, which may lead to an inconsistent, unsatisfying, and/or undesirable user experience. Moreover, variability in aerosol production correlates to variability in dosage, which may be of particular concern in medicinal applications.

Saturation of the wick may be impeded by a number of factors including, for example, negative pressure that forms in the cartridge as the cartridge is depleted of the vaporizable material, high viscosity of the vaporizable material such as various cannabis oils, low surface tension of the vaporizable material, a low capillary force of the wick, and/or the like. These factors may, individually or in combination, affect both the rate of wicking (e.g., vaporizable material flowing to a region of the wick exposed to heat from the heating element) and the release of air bubbles in the wick (which flow in the opposite direction as the vaporizable material) as described in detail below.

Aspects of the current subject matter provide for improved wick saturation by addressing the described limiting factors, including reducing the viscosity of the vaporizable material and aiding in the release of the air bubbles that form and are trapped in the wick.

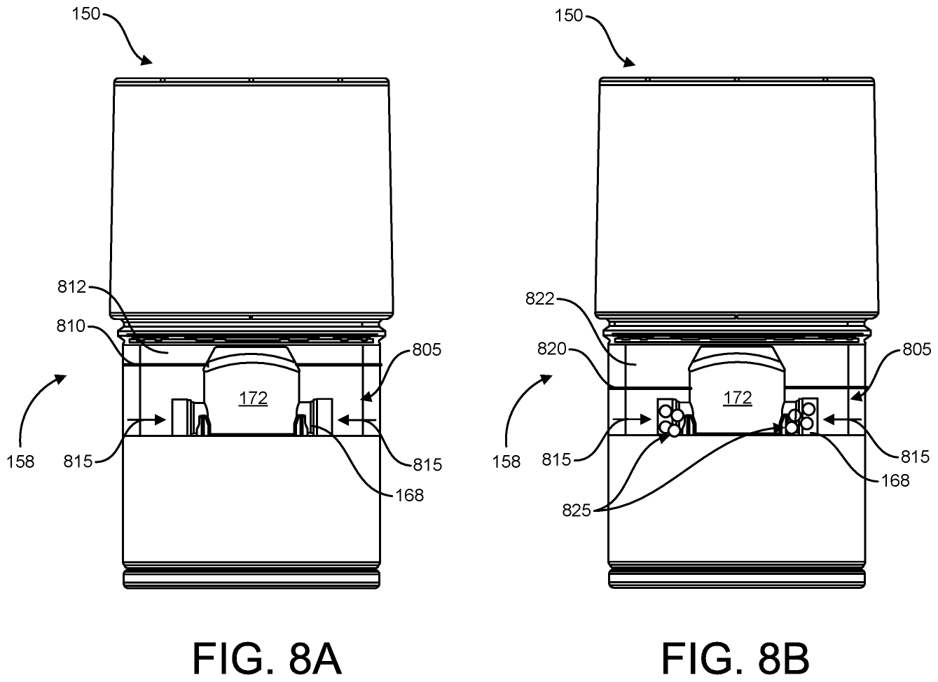

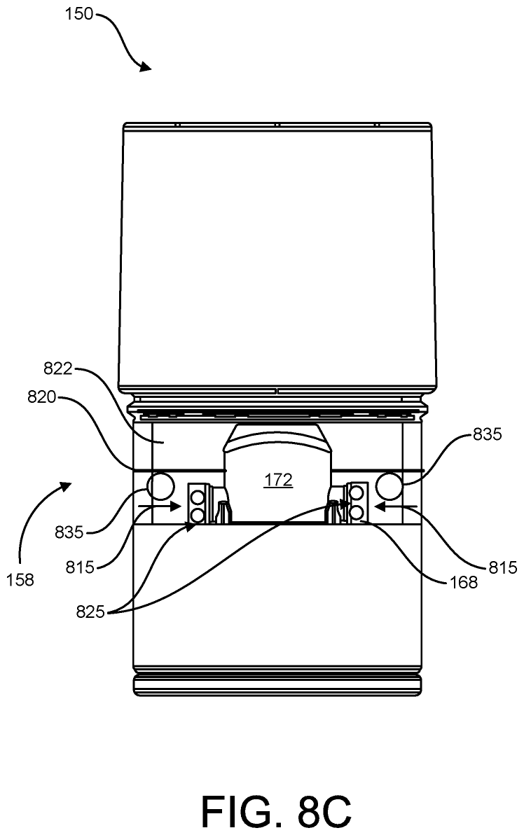

FIG. 8A-FIG. 8C illustrate pressure aspects related to the cartridge 150 and the wick 168 consistent with implementations of the current subject matter. Shown in FIG. 8A-FIG. 8C is the cartridge 150 including the central cannula 172, that defines the vaporization chamber, that extends through the reservoir 158. As described in detail with reference to FIG. 6, the central portion of the wick 168 may be positioned within the vaporization chamber, while the opposing ends of the wick 168 are positioned outside the vaporization chamber within the internal volume of the reservoir 158.

In FIG. 8A, a vaporizable material 805 is filled to a first level 810. A capillary force, indicated by arrows 815, pulls the vaporizable material 805 from the reservoir 158 axially toward the central portion of the wick 168 (e.g., the central portion 1060 of the wick 168 as shown in and described with reference to FIG. 6). A first volume 812 of air or other gas represents the volume in the reservoir 158 that is contained above the first level 810. The first volume 812 is at approximately ambient atmospheric pressure.

FIG. 8B depicts the cartridge 150 when a portion of the vaporizable material 805 is consumed by being drawn to the center of the wick 168 inside the central cannula 172, thereby lowering the vaporizable material to a second level 820. A second volume 822 represents the volume in the reservoir 158 that is contained above the second level 820. The amount of air or other gas in the second volume 822 has not changed from the amount of air or gas in the first volume 812, but as the second volume 822 is larger than the first volume 812, a pressure difference exists between the second volume 822 and the first volume 812, which was at approximately ambient atmospheric pressure (e.g., there is a pressure drop in the second volume 822). To relieve the pressure difference, air (shown in the form of air bubbles 825) may be naturally pulled through the wick 168 toward the reservoir 158. At the same time, the capillary force 815 continues to draw the vaporizable material 805 from the reservoir 158 toward the central portion of the wick 168. The release of the air bubbles 825 is impeded by the opposing capillary force 815.

At some time after the naturally occurring circumstances depicted in FIG. 8B, the pressure differential is relieved, and released air bubbles 835 are released from the wick 168 into the reservoir 158, as shown in FIG. 8C. At this point, there may still be a need to release the air bubbles 825 that naturally remain at end regions of the wick 168, as the continued presence of the air bubbles 825 impedes the flow of the vaporizable material into the wick 168.

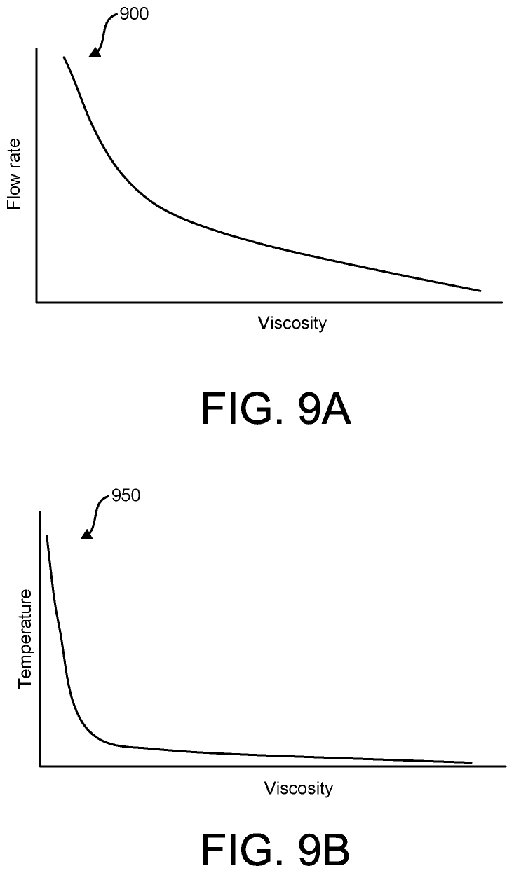

FIG. 9A is a graph 900 illustrating viscosity properties related to flow rate. In particular, the graph 900 illustrates the principle that as viscosity increases, flow rate within the reservoir 158 and along the wick 168 is decreased; that is, a more viscous material flows more slowly than a less viscous material. FIG. 9B is a graph 950 illustrating the principle that as temperature increases, viscosity decreases. Thus, it follows that a higher flow rate can be achieved with a material that is less viscous, which may result from increasing the temperature of the material. The flow rate-viscosity and temperature-viscosity principles illustrated in FIG. 9A and FIG. 9B, respectively, are generally applicable across a variety of vaporizable materials.

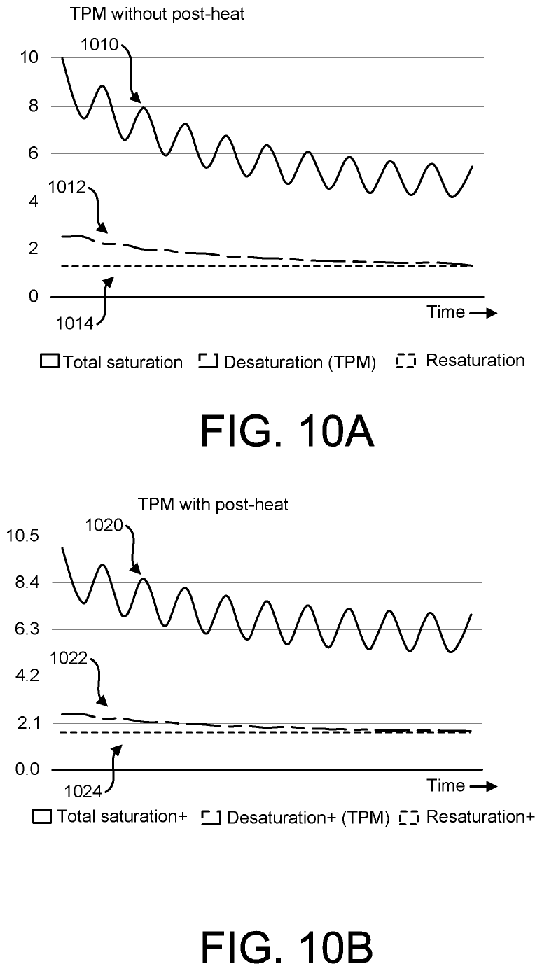

FIG. 10A and FIG. 10B illustrate total particulate matter (also referred to as TPM) properties (where total particulate matter refers to an amount of the vaporizable material removed, e.g., the total mass of the aerosol generated) with respect to time and heat of the wick 168 of the vaporizer device 100 consistent with implementations of the current subject matter. FIG. 10A illustrates total saturation 1010, desaturation 1012, and re-saturation 1014 of the wick 168 versus time in which heat is not applied, via for example the heating coil 167, after a user puffs on the mouthpiece 152 of the vaporizer device 100. Re-saturation, consistent with implementations of the current subject matter, refers to the total mass of the vaporizable material held by the wick 168 for puffs subsequent to the first puff; for example, the amount of the total mass of the vaporizable material that re-saturates the wick 168 after the first puff. For example, saturation may refer to the initial amount of the total mass of the vaporizable material held by the wick 168, and re-saturation may refer to the subsequent amount of the total mass of the vaporizable material held by the wick 168 after the first puff.

1010 represents the total mass of the vaporizable material held by the wick 168 in the absence of the improvements described herein. 1012 represents the amount of vaporizable material that may potentially be aerosolized from the wick 168, as a function of the saturation state 1010 of the wick 168. 1014 represents the rate of re-saturation in the absence of the improvements described herein, which, in ideal form is a constant. FIG. 10B illustrates total saturation 1020, desaturation 1022, and re-saturation 1024 of the wick 168 versus time in which heat is applied, via for example the heating coil 167, after a user puffs on the mouthpiece 152 of the vaporizer device 100. 1020 represents the total mass of the vaporizable material held by the wick 168 when applying the implementations of the current subject matter. 1022 represents the amount of vaporizable material that may potentially be aerosolized from the wick 168, as a function of the saturation state 1020 of the wick. 1024 represents the rate of re-saturation when applying the implementations of the current subject matter, which, in ideal form is a constant. FIG. 10A and FIG. 10B demonstrate that total particulate matter of the wick 168 is improved if heat is applied after user puffing of the vaporizer device 100.

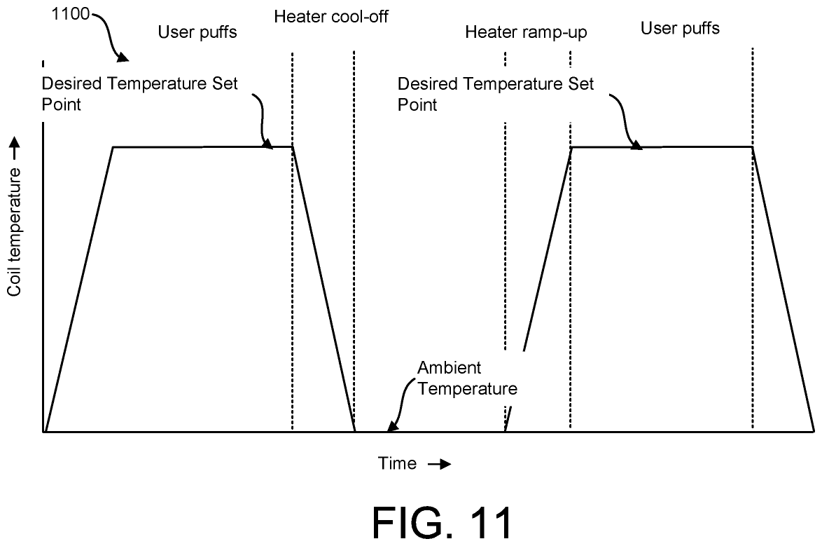

FIG. 11 illustrates, via graph 1100, temperature properties with respect to time of the heating coil 167. As shown, when a user puffs on the vaporizer device 100, the temperature of the heating coil 167 is increased to a desired temperature set point. After the user puff, there is a cool-down period during which the temperature of the heating coil 167 decreases back to ambient temperature. After a period of time, the user may puff again on the vaporizer device 100, causing the temperature of the heating coil 167 again to be increased to the desired temperature set point. Again, after the user puff, the temperature of the heating coil 167 is lowered to the ambient temperature. Although the graph 1100 depicts a linear relationship between the temperature of the heating coil 167 and time, the relationship between the two parameters is not limited to a purely linear relationship. For example, more rounded slopes may exist in the relationship between the temperature of the heating coil 167 and time.

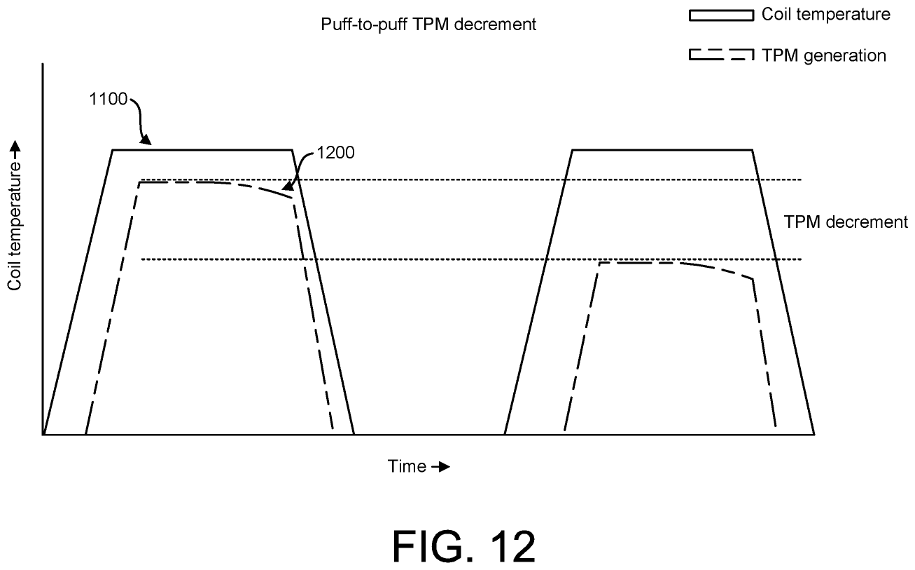

FIG. 12 illustrates, via graph 1200, total particulate matter generation with respect to time, based on the temperature of the heating coil depicted in the graph 1100. As shown, for a first puff, the total particulate matter of the wick 168 increases as the temperature of the heating coil 167 increases, then reaches a near steady state before decreasing when the puff ends and the temperature of the heating coil 167 is decreased. At some time later, in a subsequent puff at the same temperature set point and of the same duration, less total particulate matter is generated due to the wick 168 being desaturated. This difference in total particulate matter generation between the first puff and the later puff may be referred to as total particulate matter decrement.

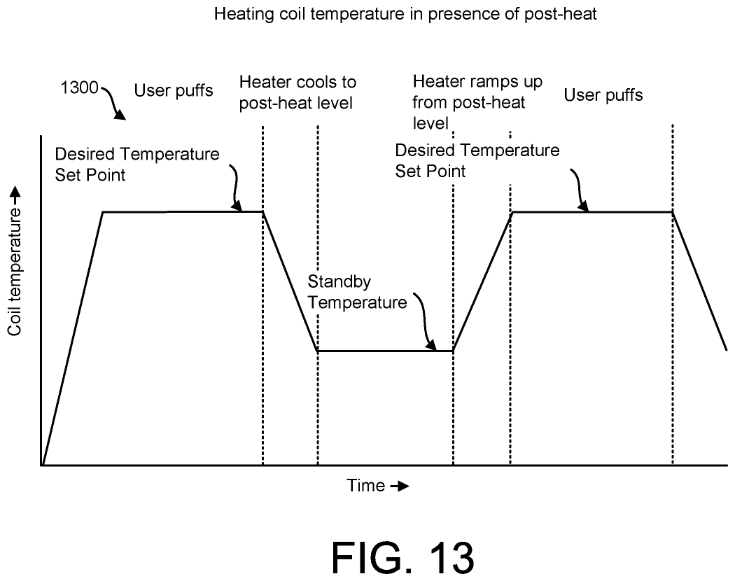

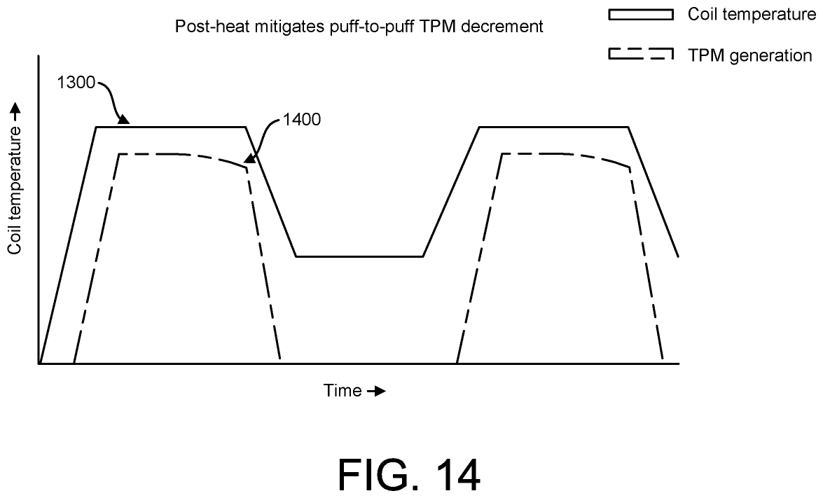

FIG. 13 and FIG. 14 illustrate the heating coil 167 temperature properties (graph 1300 in FIG. 13) and the wick 168 total particulate matter properties (graph 1400 in FIG. 14) with the temperature of the heating coil 167 being maintained in between puffs at a post-heat or standby level that is higher than the ambient temperature consistent with implementations of the current subject matter. As shown in graph 1400 in FIG. 14, when the heating coil 167 is maintained at the post-heat or standby level, total particulate matter decrement is substantially reduced for the subsequent puff. That is, compared to a situation in which the heating coil 167 is lowered to the ambient temperature between puffs, as shown in FIG. 11 and FIG. 12, when the heating coil 167 is instead maintained at a higher in-between puff temperature, the generation of total particulate matter is improved, increasing the saturation level of the wick 168.

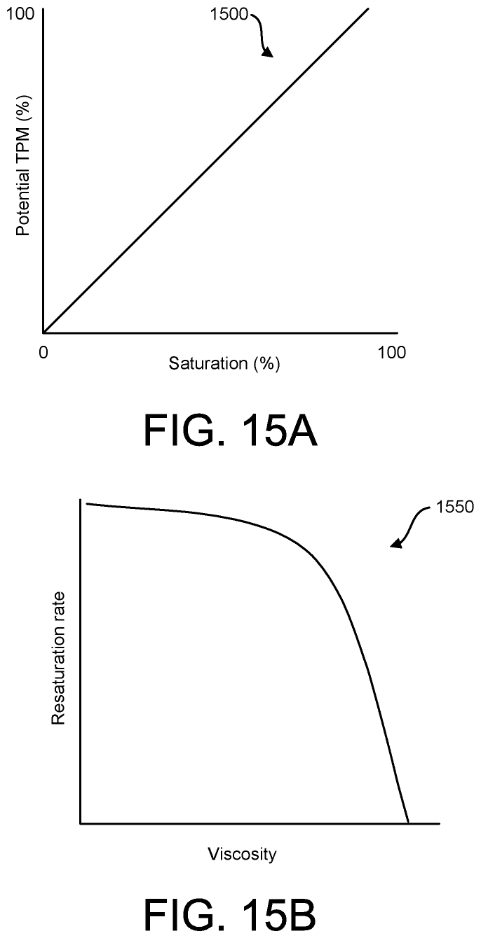

FIG. 15A and FIG. 15B illustrate saturation properties of the wick 168 with respect to total particulate matter and viscosity consistent with implementations of the current subject matter. In particular, as reflected by graph 1500 in FIG. 15A, it can be seen that the saturation of the wick 168 drives aerosol production. That is, as saturation decreases, potential total particulate matter decreases. Graph 1550 of FIG. 15B illustrates that re-saturation rate is inversely correlated to viscosity. That is, the re-saturation rate for the wick 168 decreases as viscosity is increased.

According to implementations of the current subject matter, a mechanical agitation, such as a vibration or pulsation, may be applied to the cartridge 150 to aid in releasing of the formed and trapped air bubbles (e.g., the air bubbles 825 that are formed and trapped in the wick 168 as shown in FIG. 8B). The applied mechanical agitation serves to counteract the capillary force 815 that is impeding the release of the air bubbles 825 into the reservoir 158. That is, the applied mechanical agitation causes the air bubbles 825, which are naturally being pulled through the wick 168 toward the reservoir 158, to be expelled from the wick 168 into the reservoir 158. This action increases wick saturation due to the air bubbles 825 no longer occupying space along the wick 168 (e.g., after the release of the air bubbles 825, there is more volume along the wick 168 to be saturated with the vaporizable material 805 since the air bubbles 825 are released). Moreover, the applied mechanical agitation may encourage or increase the formation of the air bubbles 825, the release of which increases saturation of the wick 168.

The mechanical agitation may be supplied by the haptics system 144 contained in the vaporizer body 110, as described with reference to FIG. 2, FIG. 4A, and FIG. 4B. Consistent with implementations of the current subject matter, the haptics system 144 may be positioned in various other positions or arrangements, such as at a different portion of the vaporizer body 110 or within the cartridge 150. As previously described, the haptics system 144 may include an actuator, a linear resonant actuator (LRA), an eccentric rotating mass (ERM) motor, or the like that provide haptic feedback such as a vibration. The controller 128 may signal the haptics system 144 to vibrate. With the cartridge 150 positioned in the cartridge receptacle 114 on the vaporizer body 110, the mechanical agitation from the haptics system 144 causes vibration (or other pulse-like movement) of the cartridge 150, including the wick 168, to aid in releasing the trapped air bubbles 825. In additional implementations, the mechanical agitation may be provided through user action, for example, shaking or tapping the vaporizer device 100 in response to an alert or instructions from the vaporizer device 100 (e.g., a particular sound or series of sounds, a particular pattern of lights, etc.) or from the user device 305 in communication with the vaporizer device 100.

Consistent with additional implementations of the current subject matter, to reduce the resistance (e.g., the capillary force 815) to aid in the release of the air bubbles 825, a standby (e.g., post-heat level) temperature setting is applied to the heating coil 167 and may be applied in between user puffs. The standby temperature setting may include one or more settings to achieve a standby temperature of the heating coil 167. For example, the standby temperature setting may include a power or energy setting or amount to apply to the heating coil 167 to achieve the standby temperature. The standby temperature setting may be correlated with the standby temperature by, for example, identifying or indicating operational parameters or settings for the vaporizer device 100 such that the heating coil 167 is heated to the standby temperature. The application of the standby temperature setting, consistent with implementations of the current subject matter, may reduce the viscosity of the vaporizable material 805 along the length of the wick 168, which allows for the air bubbles 825 to be more easily expelled from the wick 168. The standby temperature, which may be defined by or correlated with the standby temperature setting, may be greater than the ambient temperature but less than the vaporization temperature (or temperature set point). For example, the standby temperature setting may provide or result in the standby temperature of the heating coil 167 that is lower than the vaporization temperature but higher than the ambient temperature. In some implementations, the standby temperature setting is applied by the heater control circuitry 130 described above with reference to FIG. 7. In some implementations, a heating of the reservoir 158 may be applied to further aid in reducing the viscosity of the vaporizable material.

Consistent with implementations of the current subject matter, various characteristics or aspects of each of the mechanical agitation and the standby temperature setting are defined to achieve desired wick saturation properties as described further herein. The various characteristics or aspects may be applied by the vaporizer device 100 as settings. For example, the vaporizer device 100 may apply one or more mechanical agitation settings and/or one or more standby temperature settings, the settings indicative or representative of the characteristics determined to achieve the desired wick saturation properties. Some examples of the characteristics and types of characteristics are described further herein, but the wick saturation functionality in accordance with implementations of the current subject matter is not limited to these particular characteristics.

The mechanical agitation and/or the use of the standby temperature setting may be signaled to occur in between each user puff or at a predetermined schedule or iteration; for example, after the tenth puff and in between each puff thereafter; after a particular puff that exceeds a predetermined period of time; after every other puff; after every third puff; or various other schedules or iterations or combinations thereof. In some implementations, the mechanical agitation and/or the use of the standby temperature setting may depend on the viscosity of the vaporizable material and other factors as further described herein. Characteristics of the mechanical agitation and the standby temperature setting may vary and may also depend on the viscosity of the vaporizable material and other factors as further described herein.

Characteristics of the mechanical agitation may include for example frequency, duty cycle, and pattern such as time duration and intensity. A plurality of profiles, for example haptics profiles, with specific characteristics defining the mechanical agitation, al so including the schedule of occurrence for use of the mechanical agitation, may be defined and applied by signaling from the controller 128 to the haptics system 144. Similarly, characteristics of the standby temperature setting, such as the standby temperature itself, the schedule of occurrence for use of the standby temperature setting, and length of time for the standby temperature setting to be applied, may be applied by signaling from the controller 128 and/or the heater control circuitry 130 to the heating coil 167.

The use and the characteristics of the mechanical agitation, as well as the use and the characteristics of the standby temperature setting, may be based on a number of factors, such as user preferences or definitions, the type of the vaporizable material being consumed, properties (such as viscosity) of the vaporizable material, the amount of the vaporizable material remaining in the cartridge 150, the age of the vaporizable material, ambient characteristics (e.g., temperature, pressure, altitude, humidity, etc.), characteristics internal to the cartridge (e.g., temperature, pressure), and/or use data (e.g., the number of puffs that have been taken from the cartridge 150, the duration of the puffs taken, when the puffs were taken, time between successive puffs, and/or the amount of total particulate matter generated by the puffs taken). For example, higher viscosity materials may typically require a standby temperature setting that achieves a higher standby temperature for a longer period of time and/or with more or greater mechanical agitation. As another example, the lower the ambient pressure, the higher the standby temperature and the more mechanical agitation that may be required. The lower the ambient temperature, the higher the standby temperature that may be required. The higher the altitude, the lower the ambient pressure, the more mechanical agitation that may be required. As the number of puffs increases, the composition of the vaporizable material changes, possibly requiring a higher standby temperature. The more puffs taken in a single sitting, the greater the requirement for re-saturation through a combination of the mechanical agitation and/or the standby temperature setting. In situations in which the previous puff was of a longer than normal or average duration, a standby temperature setting indicative of a higher standby temperature for a longer period of time may be required to achieve greater re-saturation. The more puffs taken in a single sitting, the greater the requirement for re-saturation through a combination of agitation and temperature. The older the cartridge 150, potentially the higher the viscosity of the vaporizable material, resulting in a higher standby temperature and/or more or greater mechanical agitation to achieve greater re-saturation.

The characteristics of the mechanical agitation and the characteristics of the standby temperature setting for particular conditions and/or parameters (e.g., a particular type of vaporizable material, particular ambient conditions, etc.) may be based on experimental data and/or past use data. For example, for a type of vaporizable material, a particular standby temperature setting or a range of standby temperature settings may be determined to adequately increase the re-saturation of the wick 168. Other factors relating to the mechanical agitation and the standby temperature setting may be determined. In some implementations, the characteristics that adequately increase the re-saturation of the wick 168 may be those that provide for the total particulate matter generation for second and subsequent puffs to be equal to the total particulate matter generation for the first puff. In some implementations, the characteristics may be those that provide for the total particulate matter generation for the second and subsequent puffs to be within a predefined range of the total particulate matter generation for the first puff; for example, a total particulate matter generation of at least 80%, 85%, 90%, or 95% of the total particulate matter generation for the first puff. In some implementations, the perception of a user may be used to determine if saturation is sufficient. For example, the user may provide feedback through the app running on the user device 305 to indicate if the amount of vapor produced for one or more previous puffs was sufficient. If the user provides an indication that the amount of vapor produced is insufficient, the mechanical agitation and/or the standby temperature setting features consistent with implementations of the current subject matter may be applied. Similarly, the user may indicate that too much vapor was produced, and the mechanical agitation and/or the standby temperature setting features may be reduced and/or not used.

The use and the characteristics of the mechanical agitation, as well as the use and the characteristics of the standby temperature setting, may in some examples be user configurable and/or adjustable. For example, a user may select a feature through the app running on the user device 305 to enable or disable the mechanical agitation feature and/or the standby temperature setting feature. Additionally, a user may be able to, through the app, define a desired haptics profile, which may be device specific or cartridge specific. In other instances, the characteristics of the mechanical agitation and the standby temperature setting are pre-established and/or determined based on information available to the vaporizer device 100 and/or the user device 305. With respect to user configurability and adjustability, the user may be able to specify certain constraints or restrictions surrounding the use of wick re-saturation functionality including the mechanical agitation and the standby temperature setting. For example, the user may specify that the re-saturation functionality should not be used when a battery level of the vaporizer device 100 is at or below a certain level. This may be useful in conserving battery life for the user.

Further with respect to conservation of battery life of the vaporizer device 100 consistent with implementations of the current subject matter, variations of the wick re-saturation functionality may be applied to optimize power usage. For example, a lower standby temperature (defined by or correlated with the standby temperature setting) may be applied, a shorter duration for the standby temperature or the lower standby temperature may be used, and/or a variation of the haptics profile that uses less power (e.g., lower amplitude, reduced duty cycle, or reduced frequency) may be applied. In some implementations, variations of wick re-saturation characteristics may be those that provide for the total particulate matter generation to be of a certain value; for example, a total particulate matter generation of at least 75%, 80%, 85%, etc. of the total particulate matter generation for the first puff. In some implementations, variations of wick re-saturation characteristics may be defined as a percentage of the defined and/or determined wick re-saturation characteristics; for example for battery life conservation, battery conserving wick re-saturation characteristics may be 50%, 55%, 60%, 65%, 70%, 75%, 80%, 85%, 90%, or 95% of the defined and/or determined wick re-saturation characteristics. In some implementations, the characteristics of the mechanical agitation may be altered while those of the standby temperature setting are not altered. In some implementations, the characteristics of the standby temperature setting may be altered while those of the mechanical agitation are not altered. In some implementations, the characteristics of the mechanical agitation and the characteristics of the standby temperature setting are altered at varying degrees for battery life conservation.

Consistent with some implementations of the current subject matter, differences in viscosity of the vaporizable material may need to be taken into account for application of the mechanical agitation and the standby temperature setting. For example, vaporizable materials have different viscosities depending on such factors as chemical composition, age, and ambient properties, and such factors may need to be accounted for to achieve wick re-saturation consistent with implementations of the current subject matter. In instances in which information about the cartridge 150 and the vaporizable material is not available to the vaporizer device 100 (for example, the type of vaporizable material is not known or identifiable by the vaporizer device 100), implementations of the current subject matter may use or establish a maximum viscosity value such that a re-saturation time and temperature adequately apply to all types or a subset of types of vaporizable materials having a viscosity less than or equal to the defined maximum viscosity. Similarly, a predefined haptics profile that is applicable to all types or a subset of types of vaporizable materials may be applied. In some implementations, the applied mechanical agitation and standby temperature setting may be based on a vaporizable material with a high viscosity, such that adequate saturation of the wick 168 is achieved even if the particular vaporizable material has a lower viscosity.

In instances in which information about the cartridge 150 and/or the vaporizable material is available (for example, from a user selection of the type of cartridge/vaporizable material via the app, from an app setting, from a remote database, or from cartridge information being read by the vaporizer body 110 from the cartridge 150 in accordance with the NFC techniques disclosed herein), the viscosity of the particular vaporizable material may be read from a look-up table (stored, for example, in the memory 146 or otherwise accessible to the controller 128 (e.g., through signaling with a server or other remote device as described with reference to FIG. 3)). The look-up table and/or the NFC data may include the haptics profile that corresponds to the particular viscosity and/or vaporizable material. As previously described, the haptics profiles may include characteristics defining the mechanical agitation. The look-up table and/or the NFC data may also include the schedule of occurrence for use of the mechanical agitation, characteristics of the standby temperature setting, the schedule of occurrence for use of the standby temperature setting, and the length of time for the standby temperature setting to be applied.

In some implementations, a linear relationship between viscosity and temperature may be used to determine the standby temperature setting and period of time to apply the standby temperature setting. In some implementations, an algorithmic function may be used to determine the standby temperature setting, duration of heating, and/or intensity and duration of the mechanical agitation, based on factors including ambient temperature, pressure, composition of the material, and/or the like. This algorithm may be built using experimental data, and may be subject to refinement and revision, for example using software and/or firmware updates.

Consistent with implementations of the current subject matter, the NFC data read by the vaporizer body 110 may include the relevant re-saturation characteristics to be applied (the haptics profile, the standby temperature setting, the schedules, the length of time, etc.).

In some implementations, the viscosity may be determined or adjusted based on a number of factors available to the vaporizer body 110. For example, the controller 128 and/or the user device 305 may take into account any of the following to adjust a value of the viscosity read from the look-up table or to adjust any of the re-saturation characteristics to be applied: user preferences or definitions, the type of the vaporizable material being consumed, properties (such as viscosity) of the vaporizable material, the amount of the vaporizable material remaining in the cartridge 150, the age of the vaporizable material, ambient characteristics (e.g., temperature, pressure, altitude, humidity, etc.), characteristics internal to the cartridge 150 (e.g., temperature, pressure), characteristics of the cartridge 150 (e.g., the model number, version number, and/or date of manufacture), and/or use data (e.g., the number of puffs that have been taken from the cartridge 150, the duration of the puffs taken, when (e.g., time and/or date) the puffs were taken, time between successive puffs, and/or the estimated amount of total particulate matter generated by the puffs taken).

Consistent with additional implementations of the current subject matter, differences in material and properties of the wick 168 and/or material and properties of the heating coil 167 may need to be taken into account for application of the mechanical agitation and the standby temperature setting. In particular, different wick materials have different magnitudes of capillary effects, which affects the time, temperature, and other properties to achieve wick re-saturation. In instances in which information about the wick 168 and the heating coil 167 is not available to the vaporizer device 100, implementations of the current subject matter may use or establish re-saturation properties (e.g., the characteristics of the mechanical agitation and the standby temperature setting) that adequately apply to all types, a majority of types, or a subset of types of wicks 168 and/or heating coils 167 and/or to a subset of wicks 168 and/or heating coils 167.

In instances in which information about the wick 168 and the heating coil 167 is available (for example, from a user selection of the type of cartridge via the app, from an app setting, from a remote database, or from cartridge information being read by the vaporizer body 110 from the cartridge 150 in accordance with the NFC techniques disclosed herein), the re-saturation characteristics to be applied may be read from a look-up table or directly from the tag 164.

Consistent with implementations of the current subject matter, a determination may be done to indicate if the disclosed wick re-saturation techniques are necessary or would be beneficial to aid in aerosol production. For example, factors including rate of temperature increase of the heating coil 167, energy required to achieve the temperature increase of the heating coil, and/or the like may be used to identify energy consumption factors. A large energy consumption value, compared to a predetermined or predefined baseline energy consumption value, may serve as an indication that wick saturation is needed; while a small energy consumption value, compared to the predetermined or predefined baseline energy consumption value, may serve as an indication that wick saturation is adequate. In some implementations, if the energy consumption value is greater than a predetermined amount from the baseline energy consumption value, wick re-saturation may be needed and the wick re-saturation characteristics consistent with implementations of the current subject matter may be applied. If the energy consumption value is less than a predetermined amount from the baseline energy consumption value, current wick saturation may be deemed as being adequate and wick re-saturation not necessary. The predetermined amounts for identifying if wick re-saturation is necessary may be user-defined, manufacturer-defined, cartridge-specific (e.g., based on the type of vaporizable material, characteristics of the wick 168, and/or characteristics of the heating coil 167), and/or vaporizer device-specific. Additional factors such as ambient conditions and usage (e.g., amount of time since last puff) may also be taken into account to determine if wick re-saturation is needed.

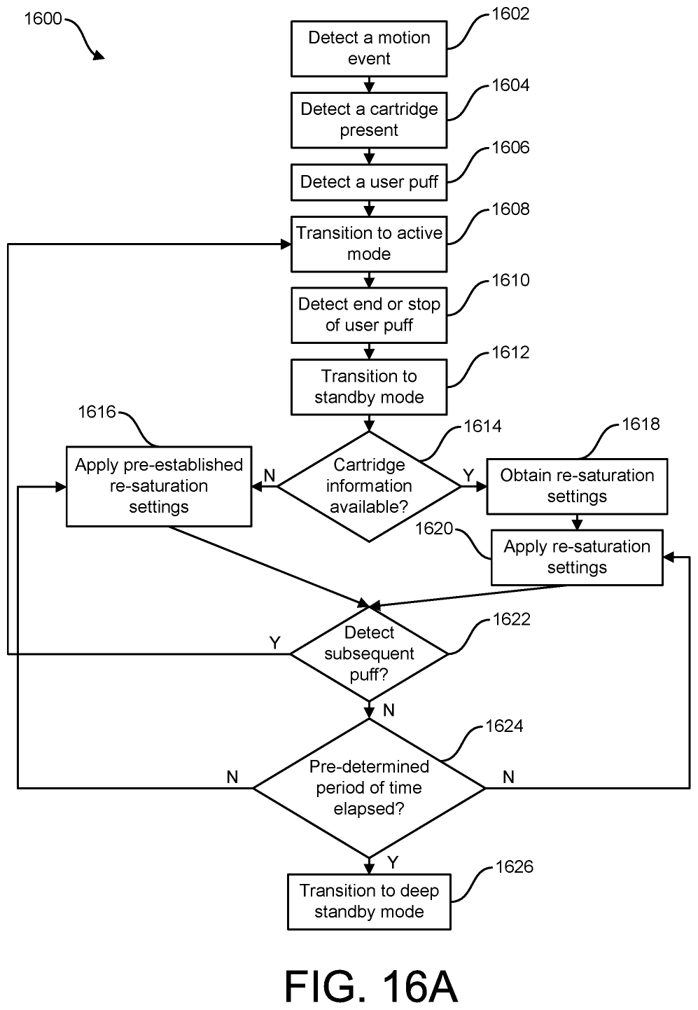

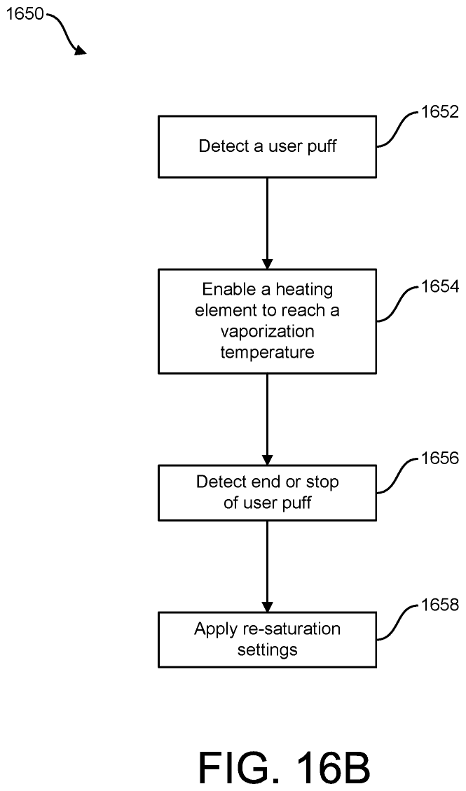

FIG. 16A depicts a flowchart illustrating a process 1600 for operating the vaporizer device 100 consistent with implementations of the current subject matter. In some example embodiments, the vaporizer device 100, for example, the controller 128, may perform the process 1600 to transition between different modes of operations including, for example, an active mode, a standby mode, and/or a deep standby mode.

At 1602, the vaporizer device 100 may detect a motion event. As noted, the vaporizer device 100 may include the accelerometer 139 (and/or other motion sensors, capacitive sensors, flow sensors, strain gauge(s), and/or the like) capable of detecting movement of the vaporizer body 110 including, for example, tapping, rolling, and/or any other deliberate movements. Such movements may be indicative of user interaction with the vaporizer device 100 and may therefore be interpreted, for example, by the controller 128, as one or more predefined user commands including, for example, a user command to gradually increase the temperature of the heater 166 before the user begins using the vaporizer device 100 and/or while the user is using the vaporizer device 100.

At 1604, the vaporizer device 100 may detect that the cartridge 150 is present in the cartridge receptacle 114 on the vaporizer body 110 of the vaporizer device 100. In some instances, one or more output signals from the heater control circuitry 130 to the controller 128 may indicate whether the cartridge 150 is present in or absent from the cartridge receptacle 114 in the vaporizer body 110 of the vaporizer device 100.

At 1606, the vaporizer device 100 may detect a user puff on the mouthpiece 152 of the vaporizer device 100. In some instances, puff detection may be based on signals to the controller 128 from the pressure sensor 137 and/or the ambient pressure sensor 138. In some instances, other forms of puff detection may be utilized, such as capacitance sensing.

At 1608, the vaporizer device 100 may respond to the puff detection by at least transitioning to the active mode in which the heating coil 167 is activated to reach a desired or selected vaporization temperature. In some instances, the heater control circuitry 130 or variations thereof may be utilized to reach the desired or selected vaporization temperature.

At 1610, the vaporizer device 100 may detect that the user puff has ended or stopped (e.g., the user has stopped mid-puff). This detection may be based on signals to the controller 128 from the pressure sensor 137 and/or the ambient pressure sensor 138. Other or additional detection methods may also be utilized in some instances.

At 1612, the vaporizer device 100 may respond to the end of puff detection by at least transitioning to a standby mode. Following transition into the standby mode, at 1614, the vaporizer device 100 may determine if information relevant to the cartridge 150 is accessible. At 1616, following a determination that cartridge information is not available (for example, the type of vaporizable material is not known or identifiable by the vaporizer device 100), the vaporizer device 100 may apply pre-established re-saturation settings. For example, a pre-established haptics profile and a pre-established standby temperature setting may be applied. The pre-established haptics profile and the pre-established standby temperature setting may be those determined to be applicable to all types or a subset of types of vaporizable materials.

At 1618, following a determination that cartridge information is available, the vaporizer device 100 may obtain applicable re-saturation settings. For example, the vaporizer device 100 may utilize one or more look-up tables and/or other available data related to the vaporizable material, the wick 168, and/or the heating coil 167 (e.g., NFC data). The one or more look-up tables and/or the NFC data may provide the vaporizer device 100 with applicable re-saturation settings to be applied, such as the haptics profile and/or the standby temperature setting including, for example, the amount of power or energy to apply to the heating coil 167 and/or the duration of time for applying the amount of power or energy to the heating coil 167. At 1620, the applicable re-saturation settings are applied.

When the vaporizer device 100 is in the standby mode, a subsequent puff may be detected (1622), in which case the vaporizer device 100 may transition to the sequence of entering the active mode.

At 1624, a determination is made as to whether a predetermined period of time has elapsed. If the predetermined period of time has not elapsed, the vaporizer device 100 may remain in the standby mode during which the pre-established re-saturation settings continue to be applied (1616) or the obtained re-saturation settings continue to be applied (1620). If however, the predetermined period of time has elapsed, the vaporizer device 100 may transition to the deep standby mode (1626). In the deep standby mode, the heating coil 167 may not receive any power for heating the vaporizable material. The predetermined period of time may be an amount of time that this is indicative of a user stopping use of the vaporizer device 100, based on for example previous user data. The predetermined period of time may also or additionally be based on battery life. For example, the vaporizer device 100 may enter the deep standby mode after a period of time has elapsed at which the battery life is at 50% (or other threshold value) or below.

It should be appreciated that the process 1600 may be an example of a process for transitioning the operation mode of the vaporizer device 100 and utilizing wick re-saturation functionality. Different processes may be implemented for transitioning the operation mode of the vaporizer device 100 and for utilizing wick re-saturation functionality.

FIG. 16B depicts a flowchart illustrating a process 1650 for operating the vaporizer device 100 consistent with implementations of the current subject matter. In some example embodiments, the vaporizer device 100, for example, the controller 128, may perform the process 1650.

At 1652, the vaporizer device 100 may detect a user puff on the mouthpiece 152 of the vaporizer device 100. In some instances, puff detection may be based on signals to the controller 128 from the pressure sensor 137 and/or the ambient pressure sensor 138. In some instances, other forms of puff detection may be utilized such as capacitance sensing.

At 1654, the vaporizer device 100 may enable a heating element (e.g., the heating coil 167) of the vaporizer device 100 to reach a desired or selected vaporization temperature. In some instances, the heater control circuitry 130 or variations thereof may be utilized to reach the desired or selected vaporization temperature.

At 1656, the vaporizer device 100 may detect that the user puff has ended or stopped (e.g., the user has stopped mid-puff). This detection may be based on signals to the controller 128 from the pressure sensor 137 and/or the ambient pressure sensor 138. Other or additional detection methods may also be utilized in some instances.

At 1658, the vaporizer device 100 may apply, in response to the detection of the end of the user puff, re-saturation settings. In some instances, the re-saturation settings may be pre-established re-saturation settings; for example, a pre-established haptics profile and a pre-established standby temperature setting to achieve a standby temperature. The pre-established haptics profile and the pre-established standby temperature setting may be those determined to be applicable to all types or a subset of types of vaporizable materials. In some instances, the vaporizer device 100 may utilize one or more look-up tables and/or other available data related to the vaporizable material, the wick 168, and/or the heating coil 167 (e.g., NFC data). The one or more look-up tables and/or the NFC data may provide the vaporizer device 100 with applicable re-saturation settings to be applied, such as the haptics profile and/or the standby temperature setting including, for example, the amount of power or energy to apply to the heating coil 167 and/or the duration of time for applying the amount of power or energy to the heating coil 167. The application of the re-saturation settings may include applying a standby temperature setting such that the heating coil 167 is heated to a standby temperature and applying a mechanical agitation by a haptics system of the vaporizer device 100. The re-saturation settings may, consistent with implementations of the current subject matter, define one or more characteristics of the standby temperature setting and one or more characteristics of the mechanical agitation.

Aspects of the current subject matter provide for improved wick saturation in a vaporizer device by applying re-saturation settings between user puffs. The re-saturation settings include a mechanical agitation by a haptics system of the vaporizer device and/or a standby temperature setting such that a heating element of the vaporizer device is at a standby temperature to aid in relieving negative pressure that forms in the cartridge as the cartridge is depleted of the vaporizable material and to decrease the viscosity of the vaporizable material, thereby resulting in improved wick saturation. By improving wick saturation, aerosol production is increased, leading to a more consistent and expected user experience.

In some examples, the vaporizable material may include a viscous liquid such as, for example a cannabis oil. In some variations, the cannabis oil comprises between 0.3% and 100% cannabis oil extract. The viscous oil may include a carrier for improving vapor formation, such as, for example, propylene glycol, glycerol, medium chain triglycerides (MCT) including lauric acid, capric acid, caprylic acid, caproic acid, etc., at between 0.01% and 25% (e.g., between 0.1% and 22%, between 1% and 20%, between 1% and 15%, and/or the like). In some variations the vapor-forming carrier is 1,3-Propanediol. A cannabis oil may include a cannabinoid or cannabinoids (natural and/or synthetic), and/or a terpene or terpenes derived from organic materials such as for example fruits and flowers. For example, any of the vaporizable materials described herein may include one or more (e.g., a mixture of) cannabinoid including one or more of: CBG (Cannabigerol), CBC (Cannabichromene), CBL (Cannabicyclol), CBV (Cannabivarin), THCV (Tetrahydrocannabivarin), CBDV (Cannabidivarin), CBCV (Cannabichromevarin), CBGV (Cannabigerovarin), CBGM (Cannabigerol Monomethyl Ether), Tetrahydrocannabinol, Cannabidiol (CBD), Cannabinol (CBN), Tetrahydrocannabinolic Acid (THCA), Cannabidioloc Acid (CBDA), Tetrahydrocannabivarinic Acid (THCVA), one or more Endocannabinoids (e.g., anandamide, 2-Arachidonoylglycerol, 2-Arachidonyl glyceryl ether, N-Arachidonoyl dopamine, Virodhamine, Lysophosphatidylinositol), and/or a synthetic cannabinoids such as, for example, one or more of: JWH-018, JWH-073, CP-55940, Dimethylheptylpyran, HU-210, HU-331, SR144528, WIN 55,212-2, JWH-133, Levonantradol (Nantrodolum), and AM-2201. The oil vaporization material may include one or more terpene, such as, for example, Hemiterpenes, Monoterpenes (e.g., geraniol, terpineol, limonene, myrcene, linalool, pinene, Iridoids), Sesquiterpenes (e.g., humulene, farnesenes, farnesol), Diterpenes (e.g., cafestol, kahweol, cembrene and taxadiene), Sesterterpenes, (e.g., geranylfarnesol), Triterpenes (e.g., squalene), Sesquarterpenes (e.g., ferrugicadiol and tetraprenylcurcumene), Tetraterpenes (lycopene, gamma-carotene, alpha- and beta-carotenes), Polyterpenes, and Norisoprenoids. For example, an oil vaporization material as described herein may include between 0.3-100% cannabinoids (e.g., 0.5-98%, 10-95%, 20-92%, 30-90%, 40-80%, 50-75%, 60-80%, etc.), 0-40% terpenes (e.g., 1-30%, 10-30%, 10-20%, etc.), and 0-25% carrier (e.g., medium chain triglycerides (MCT)).

In any of the oil vaporizable materials described herein (including in particular, the cannabinoid-based vaporizable materials), the viscosity may be within a predetermined range. The range may be between, at room temperature (23° C.) about 30 cP (centipoise) and 115 kcP (kilocentipoise), between 30 cP and 200 kcP, although higher viscosities and/or lower viscosities may be implemented as well. For example, the viscosity may be between 40 cP and 113 kcP at room temperature. Outside of this range, the vaporizable material may fail in some instances to wick appropriately to form a vapor as described herein. In particular, it is typically desired that the oil may be made sufficiently thin to both permit wicking at a rate that is useful with the apparatuses described herein, while also limiting leaking (e.g., viscosities below that of ˜30 cP at room temperature might result in problems with leaking).

Although the disclosure, including the figures, described herein may described and/or exemplify these different variations separately, it should be understood that all or some, or components of them, may be combined.

Although various illustrative embodiments are described above, any of a number of changes may be made to various embodiments. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the claims.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. References to a structure or feature that is disposed “adjacent” another feature may have portions that overlap or underlie the adjacent feature.

Terminology used herein is for the purpose of describing particular embodiments only and is not intended to be limiting. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as, for example, “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings provided herein.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” “or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, are possible.

In the descriptions above and in the claims, phrases such as, for example, “at least one of” or “one or more” of may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A vaporizer device, comprising:
at least one data processor; and
at least one memory storing instructions which, when executed by the at least one data processor, cause operations comprising:
detecting an end of a user puff on a cartridge of the vaporizer device, the cartridge including a vaporizable material; and
applying, in response to the detection of the end of the user puff, one or more of a standby temperature setting to a heating element of the vaporizer device and a mechanical agitation by a haptics system of the vaporizer device;
wherein the heating element is in thermal contact with a wick configured to convey from a reservoir the vaporizable material to a part of the cartridge in which the heating element is contained, wherein the vaporizable material is vaporized by heat delivered from the heating element.

2. The vaporizer device of claim 1, wherein the instructions, when executed, cause operations further comprising:
detecting a user puff on the cartridge; and
enabling the heating element of the vaporizer device to reach a vaporization temperature.

3. The vaporizer device of claim 2, wherein the standby temperature setting provides a standby temperature of the heating element that is lower than the vaporization temperature and higher than an ambient temperature.

4. The vaporizer device of any of claims 1-3, wherein re-saturation settings define one or more characteristics of the standby temperature setting and/or one or more characteristics of the mechanical agitation.

5. The vaporizer device of claim 4, wherein the one or more characteristics of the standby temperature setting and/or the one or more characteristics of the mechanical agitation are based on properties of the vaporizable material.

6. The vaporizer device of any of claims 4-5, wherein the one or more characteristics of the standby temperature setting comprises a length of time for the standby temperature setting to be applied.

7. The vaporizer device of any of claims 4-6, wherein the one or more characteristics of the mechanical agitation comprises one or more of a defined frequency, a defined duty cycle, a defined time duration, and a defined intensity.

8. The vaporizer device of any of claims 4-7, wherein the re-saturation settings are based on a maximum viscosity value.

9. The vaporizer device of any of claims 4-8, wherein the re-saturation settings are based on user preferences, a type of the vaporizable material being consumed, properties of the vaporizable material, an amount of the vaporizable material remaining in the cartridge, an age of the vaporizable material, ambient characteristics, characteristics internal to the cartridge, use data, properties of the wick, properties of the heating element, or a combination thereof.

10. The vaporizer device of claim 9, wherein the use data comprises a number of puffs taken from the cartridge, a duration of the puffs taken, time the puffs were taken, time between successive puffs, an amount of total particulate matter generated by the puffs taken, or a combination thereof.

11. The vaporizer device of any of claims 4-10, wherein the instructions, when executed, cause operations further comprising:
receiving, through the cartridge, the re-saturation settings.

12. The vaporizer device of any of claims 4-11, wherein the re-saturation settings are determined such that a total particulate matter generation for second and subsequent puffs is within a predefined range of a first total particulate matter generation for a first puff.

13. The vaporizer device of any of claims 1-12, wherein the one or more of the standby temperature setting and the mechanical agitation are consistent with a power or battery setting defined by a user or predefined by the vaporizer device.

14. The vaporizer device of any of claims 1-13, wherein the haptics system comprises an actuator, a linear resonant actuator, an eccentric rotating mass motor, or a combination thereof.

15. The vaporizer device of any of claims 1-14, wherein the one or more of the standby temperature setting and the mechanical agitation are applied in between user puffs at a predetermined schedule of frequency.

16. The vaporizer device of any of claims 1-15, wherein the instructions, when executed, cause operations further comprising:
receiving, through a user setting, an application setting, a remote database, the cartridge, or a combination thereof, information relating to the vaporizable material.

17. The vaporizer device of claim 16, wherein the information relating to the vaporizable material comprises at least a viscosity of the vaporizable material, and wherein one or more characteristics of the standby temperature setting and/or one or more characteristics of the mechanical agitation are provided via one or more look-up tables utilizing the viscosity of the vaporizable material.

18. The vaporizer device of claim 17, wherein the one or more look-up tables are accessible through memory of the vaporizer device, a controller of the vaporizer device via signaling with one or more remote devices, or a combination thereof.

19. A method, comprising:
detecting an end of a user puff on a cartridge of a vaporizer device, the cartridge including a vaporizable material; and
applying, in response to the detection of the end of the user puff, one or more of a standby temperature setting to a heating element of the vaporizer device and a mechanical agitation by a haptics system of the vaporizer device;
wherein the heating element is in thermal contact with a wick configured to convey from a reservoir the vaporizable material to a part of the cartridge in which the heating element is contained, wherein the vaporizable material is vaporized by heat delivered from the heating element.

20. The method of claim 19, further comprising:
detecting a user puff on the cartridge; and
enabling the heating element of the vaporizer device to reach a vaporization temperature.

21. The method of claim 20, wherein the standby temperature setting provides a standby temperature of the heating element that is lower than the vaporization temperature and higher than an ambient temperature.

22. The method of any of claims 19-21, wherein re-saturation settings define one or more characteristics of the standby temperature setting and/or one or more characteristics of the mechanical agitation.

23. The method of claim 22, wherein the one or more characteristics of the standby temperature setting and/or the one or more characteristics of the mechanical agitation are based on properties of the vaporizable material.

24. The method of any of claims 22-23, wherein the one or more characteristics of the standby temperature setting comprises a length of time for the standby temperature setting to be applied.

25. The method of any of claims 22-24, wherein the one or more characteristics of the mechanical agitation comprises one or more of a defined frequency, a defined duty cycle, a defined time duration, and a defined intensity.

26. The method of any of claims 22-25, wherein the re-saturation settings are based on a maximum viscosity value.

27. The method of any of claims 22-26, wherein the re-saturation settings are based on user preferences, a type of the vaporizable material being consumed, properties of the vaporizable material, an amount of the vaporizable material remaining in the cartridge, an age of the vaporizable material, ambient characteristics, characteristics internal to the cartridge, use data, properties of the wick, properties of the heating element, or a combination thereof.

28. The method of claim 27, wherein the use data comprises a number of puffs taken from the cartridge, a duration of the puffs taken, time the puffs were taken, time between successive puffs, an amount of total particulate matter generated by the puffs taken, or a combination thereof.

29. The method of any of claims 22-28, further comprising:
receiving, through the cartridge, the re-saturation settings.

30. The method of any of claims 22-29, wherein the re-saturation settings are determined such that a total particulate matter generation for second and subsequent puffs is within a predefined range of a first total particulate matter generation for a first puff.

31. The method of any of claims 19-30, wherein the one or more of the standby temperature setting and the mechanical agitation are consistent with a power or battery setting defined by a user or predefined by the vaporizer device.

32. The method of any of claims 19-31, wherein the haptics system comprises an actuator, a linear resonant actuator, an eccentric rotating mass motor, or a combination thereof.

33. The method of any of claims 19-32, wherein the one or more of the standby temperature setting and the mechanical agitation are applied in between user puffs at a predetermined schedule of frequency.

34. The method of any of claims 19-33, further comprising:
receiving, through a user setting, an application setting, a remote database, the cartridge, or a combination thereof, information relating to the vaporizable material.

35. The method of claim 34, wherein the information relating to the vaporizable material comprises at least a viscosity of the vaporizable material, and wherein one or more characteristics of the standby temperature setting and/or one or more characteristics of the mechanical agitation are provided via one or more look-up tables utilizing the viscosity of the vaporizable material.

36. The method of claim 35, wherein the one or more look-up tables are accessible through memory of the vaporizer device, a controller of the vaporizer device via signaling with one or more remote devices, or a combination thereof.

37. A non-transitory computer readable medium storing instructions, which when executed by at least one data processor, result in operations comprising:
detecting an end of a user puff on a cartridge of a vaporizer device, the cartridge including a vaporizable material; and
applying, in response to the detection of the end of the user puff, one or more of a standby temperature setting to a heating element of the vaporizer device and a mechanical agitation by a haptics system of the vaporizer device;
wherein the heating element is in thermal contact with a wick configured to convey from a reservoir the vaporizable material to a part of the cartridge in which the heating element is contained, wherein the vaporizable material is vaporized by heat delivered from the heating element.

38. An apparatus comprising:
means for detecting an end of a user puff on a cartridge of a vaporizer device, the cartridge including a vaporizable material; and
means for applying, in response to the detection of the end of the user puff, one or more of a standby temperature setting to a heating element of the vaporizer device and a mechanical agitation by a haptics system of the vaporizer device;
wherein the heating element is in thermal contact with a wick configured to convey from a reservoir the vaporizable material to a part of the cartridge in which the heating element is contained, wherein the vaporizable material is vaporized by heat delivered from the heating element.

39. The apparatus of claim 38, further comprising means for performing any of claims 20-36.
